(19) United States
(12) Patent Application Publication
(10) Pub. No.: US 2025/0339060 A1
(43) Pub. Date: Nov. 6, 2025

(54) DEVICES, SYSTEMS AND METHODS FOR BLOOD GLUCOSE MONITORING

(71) Applicant: SOUTH KING HEALTH LIMITED, HONG KONG (CN)

(72) Inventor: Pan LI, HONG KONG (CN)

(21) Appl. No.: 18/992,948

(22) PCT Filed: Jul. 12, 2023
(86) PCT No.: PCT/CN2023/106925

§ 371 Date: Jan. 9, 2025

Related U.S. Application Data
(60) Provisional application No. 63/388,434, filed on Jul. 12, 2022.

(51) Int. Cl. A61B 5/145; A61B 5/00; A61B 5/1455

(52) U.S. Cl. CPC A61B 5/14532; A61B 5/1455; A61B 5/7267

(57) ABSTRACT

Devices, systems and methods for blood glucose monitoring. The device includes a light emitter, configured to emit light signals; a light receiver, configured to receive the reflected light signal; a controller, configured to operatively connect with the light emitter and the light receiver; and an enclosure. The light signal comprises a first light signal having a first wavelength of about 940 nm, a second light signal having a second wavelength of about 1350 nm, and/or a third light signal having a third wavelength of about 1500 nm, wherein the controller comprises an operating module, and further comprises or operatively connects with a data processing system comprising a machine learning module that analyzes the data signal to generate an output data. The devices, systems and methods are non-invasive and monitor blood glucose levels in real time with high accuracy.

### CROSS-REFERENCE TO RELATED APPLICATION

This application claims benefit under 35 U.S.C. § 119(e) of U.S. Provisional Application Ser. No. 63/388,434 filed Jul. 12, 2022, the entire contents of which is/are hereby incorporated by reference herein.

### FIELD OF INVENTION

The present application relates to devices, systems and methods for detecting and monitoring glucose levels. The present application relates to devices, systems and methods for detecting and monitoring blood glucose levels in a user with non-invasive techniques.

### BACKGROUND OF INVENTION

Diabetes is a disease that affects over 34.2 million people around the world. In order to effectively manage diabetic symptoms, people with diabetes may monitor their blood glucose levels in order to monitor corresponding insulin levels within the body.

Typically, measuring blood glucose levels in a user requires the user to repetitively draw blood from, for example, a finger in order to apply that blood to a test strip. Or, a user may be required to wear a continuous glucose monitoring device, which includes a sensor implanted into the user's interstitial fluid space in the skin. Both methods are invasive to the user.

There exists great need to establish a reliable, needleless, device, system and methods for continuously detecting and monitoring blood glucose levels in real time, which does not require such invasive procedures.

### SUMMARY OF INVENTION

Provided are devices, systems and methods for blood glucose monitoring, data processing systems, computer implemented methods, methods for processing data from devices or systems thereof, and methods for training machine learning systems for data obtained from devices or systems thereof.

In some embodiments, provided is a device for blood glucose monitoring of a user, including: (a) a light emitter, configured to emit a light signal directed to a target surface of the user, so as to generate a reflected light signal that is reflected from the target surface; (b) a light receiver, configured to receive the reflected light signals; (c) a controller, configured to operatively connect with the light emitter and the light receiver; and (d) an enclosure, configured to receive the light emitter, the light receiver and the controller. The light signal includes a first light signal having a first wavelength of about 940 nm, a second light signal having a second wavelength of about 1350, and/or a third light signal having a third wavelength of about 1500 nm. The controller includes an operating module that controls the operation of the device, and converts the reflected light signal into a digital data. The controller further includes or operatively connects with a data processing system that processes the digital data, wherein the data processing system includes a machine learning module that analyzes the data signal to generate an output data that is a blood glucose level of the user. In some embodiments, the light signal comprises one or more wavelengths selected from a range of 400 nm-2000 nm. In some embodiments, the first wavelength is 400 nm-950 nm, the second wavelength is 1000 nm-1450 nm, and the third wavelength is 1500-2000 nm.

In some embodiments, provided is a device for blood glucose monitoring of a user, including: a) a light emitter, having a plurality of near infrared LEDs, configured to emit a light signal directed to a target surface of the user, so as to generate a reflected light signal that is reflected from the target surface, respectively, wherein the light signal includes a first light signal having a wavelength of about 950 nm, a second light signal having a wavelength of about 1350 nm and a third light signal having a wavelength of about 1500 nm; b) a light receiver, having a photo-detector, configured to receive the reflected light signals; and c) a controller, configured to operatively connect with the light emitter and the light receiver; and d) an enclosure, configured to receive the light emitter, the light receiver and the controller. The controller is configured to control the light emitter to switch on and off to emit the first light signal, the second light signal and the third light signal back-to-back sequentially, such that a plurality of reflected light groups, each including the first reflected light, the second reflected light and the third reflected light obtained in each cycle are formed at a defined time interval. The controller includes a processor unit coupled with a memory that stores an executable, software program, the software program includes an operating module that controls the operation of the device, and converts individual reflected light groups into a digital data vector. The controller further includes or operatively connects with a data processing system that processes the data vector, wherein the data processing system includes a machine learning module that analyzes the data vector to generate an output data that is a blood glucose level of the user. In some other embodiments, the sequential signal sampling process is reconfigured to concurrent sampling if the a concurrent processing controller is used.

In some embodiments, provided is a system for blood glucose monitoring of a user, including: (a) a device as claimed in any one of the preceding claims; and (b) a server in electrical communication with the device.

In some embodiments, provided is a glucose monitoring system including: a wearable device capable of measuring blood glucose levels in a user, the device including: a housing body, an optical sensor including a circuit including one or multiple near infrared light emitting diodes and a receiver chip and configured to produce a voltage signal, and a processing unit configured to convert the analog voltage signal to a digital voltage signal; and computer software including algorithms producing trained neural network models capable of predicting the user's blood glucose level in real time based on the voltage signals received from the processing unit, wherein the system is configured to non-invasively measure the user's blood glucose levels in real time, wherein the trained neural network models includes a trained non-liner model and a linear model to execute the following steps: producing a class prediction probability value and a numerical value, by subjecting the voltage signals to the trained non-linear model and the linear model, respectively; classifying the class prediction probability value and the numerical value as being low, normal or high; comparing the classification results to determine if the values are consistent; and if the values are consistent, determining the output blood glucose state and the blood glucose value.

In some embodiments, provided is a method of monitoring blood glucose level, including the steps of: (i) obtaining the first reflected light, the second reflected light and the third reflected light from the device as described in any one of the preceding embodiments, or the system as described in any one of the preceding embodiments; and (ii) calculating a blood glucose level based on the first reflected light, the second reflected light and the third reflected light.

In some embodiments, provided is a method for processing data from a device or a system for blood glucose monitoring, including the steps of: a) obtaining the data vector obtained from the device as described in any one of the preceding embodiments, or the system as described in any one of the preceding embodiments; b) pre-processing the data vector; and c) analyzing the data vector by trained neural network model to produce an output data, such that a blood glucose level of the user is obtained.

In one embodiment, a glucose monitoring system includes a wearable device capable of measuring blood glucose levels in a user. In this embodiment, the device includes a housing body, an optical sensor having a circuit with one or multiple near infrared light emitting diodes and a receiver chip, wherein the sensor is configured to produce an analog voltage signal. The system may further include a Microcontroller Unit (MCU) that contains a built-in Analog-to-digital converter (ADC) converting the analog voltage signal to a digital voltage signal and that hosts computer software containing an algorithm that produces trained neural network models capable of predicting the user's blood glucose level in real time based on the digital voltage signals. In one embodiment, the system is configured to non-invasively measure the user's blood glucose levels in real time.

There are many advantages of the present disclosure. In certain embodiments, the provided devices, systems and methods are non-invasive and solved the technical hurdles related to low detection limits and selectivity of glucose measurements for existing non-invasive optical glucose monitoring (NIO GM) technologies by detecting single or multiple near-infrared (NIR) signals. For example, one single NIR signal has a wavelength of about 940-950 nm. For example, the multiple NIR signals have a specific combination of three wavelengths of about 940 nm, about 1350 nm and about 1500 nm. With the multiple wavelength detection, NIR signals at multiple wavelengths can provide a vector of differentiation for inferring the blood glucose absorption factor.

In certain embodiments, the provided devices, systems and methods are based on non-invasive optical glucose monitoring (NIO GM) technology with the single or multiple-NIR detection using machine learning module such as deep meta-learning frameworks to solve the technical problem that the spectroscopic signals originating from glucose molecules are weak, so as to improve the signal-to-noise ratio (SNR) of the instrumentation.

In certain embodiments, the provided devices, systems and methods provide outstanding selective measurement signals over background noises relative to other components of skin, such as membranes, glycosylated structures, and soluble compounds within the ISF matrix, such as albumin, urea, amino acids, and ascorbic acid, thereby providing a robust basis for measurement accuracy. For example, the subtle differences between different components of skin can be captured and derived from the detection levels of reflected light signals of 940 nm, 1350 nm and/or 1500 nm, by the following calculation. The overall absorption factor of light signals traveling through blood is calculated as:

$$μ ( λ ) = {ε}_{1} ( λ ) {c}_{1} + {ε}_{2} ( λ ) {c}_{2} ... + {ε}_{n} ( λ ) {c}_{n} .$$

where λ is the wavelength of light, ε is the attenuation coefficient of tissues and blood components, and c is species concentration.

In certain embodiments, the provided devices, systems and methods can reduce the interference or impact of one or more of the following parameters to the results of measurement: skin pigmentation, surface roughness, skin thickness, breathing artifacts, blood flow, body movements, and ambient temperature.

In certain embodiments, the provided devices, systems and methods are based on primary (or direct) glucose sensing as well as secondary (or indirect) glucose sensing. Primary measurements involve collecting a signal derived directly from the glucose molecule, while secondary measurements involve measuring one or more parameters impacted by the concentration of glucose, such as: heart rate changes with electrocardiography; rate of red blood cell aggregation with ultrasound; blood volume dynamics with photoplethysmographic measurement of blood; dielectric properties of the skin matrix with diffuse scattering or temperature-modulated localized reflectance; and/or sudomotor dysfunction with electrochemical skin conductance and sweating asymmetry.

In certain embodiments, the provided devices, systems and methods reduce the impact of skin pigmentation on the accuracy of clinical pulse oximetry measurements, and various substances that may affect skin pigmentation, skin structure, and reflectance properties of the probing radiation include topical medications, cosmetics, sweat, cosmeceuticals, and estrogen, as well as tobacco, and alcohol.

In certain embodiments, the provided devices, systems and methods take multiple factors other than glucose (such as skin type, sweat, cholesterol, and other blood components) as a composite factor. Then, by collecting the received NIR light signal intensity at the multiple (e.g., three or more) different wavelengths and training the deep meta-learning model over data covering the key user varieties (like those in the composite factor), the impact of these factors can be learnt and hence the blood glucose level can be determined accurately.

In certain embodiments, the provided devices, systems and methods provided a deep meta-learning architecture, which involves using multiple, different machine learning/deep learning models (student-learners) to learn on the same task of glucose level prediction, respectively, and then using a meta-learning model (meta-learner) to learn how to aggregate their prediction results and obtain the final prediction result. In summary, the provided devices, systems and methods provides a meta-learner optimizes and aggregates the n basis machine learning/deep learning models. Therefore, the provided devices, systems and methods provides a deep meta-learning framework that can improve the efficiency and effectiveness of individual machine learning/deep learning algorithms and the accuracy of the data, even if the training data is limited, and/or even if there is significant variability in the data.

In certain embodiments, the provided devices, systems and methods provide a three-wavelength NIR Sensor Module (including the transmitter, photo-receiver, refractor and optical path), to capture the reflected light signals of NIR (e.g., 940 nm, 1350 nm, and 1500 nm) which contains absorption information of blood glucose as well as the other components such as skin/blood-vessel/blood-fluid. Without bound by any theory, the combinational responses of 1350 nm and 1500 nm are sensitive enough to capture the slightest difference between blood glucose and other blood components (such as cholesterol) on the spectral response. Whereas, without bound by any theory, the combination of data-streams of 940 nm and 1500 nm, will characterize the difference between blood glucose and the rest of the blood constituents.

In certain embodiments, the provided devices, systems and methods provide deep meta-learning (also referred as ‘meta deep learning’) frameworks for accurate blood glucose level determination based on data streams at multiple wavelengths (such as 940 nm/1350 nm/1500 nm). The Deep-Learning Frameworks may be provided on-line from the backend (e.g., a server), or as a plug-in component to the embedded system of the device.

### BRIEF DESCRIPTION OF FIGURES

Further embodiments of the present application can be understood with reference to the appended figures.

FIG. 1A is a schematic view of an example of the blood glucose monitoring device system.

FIG. 1B is a schematic view of the blood glucose monitoring system in use.

FIG. 1C is a schematic view of the circuit of the blood glucose monitoring device system.

FIG. 1D is a depiction of the path the light travels through the skin of the user.

FIG. 1E illustrates a block diagram describing the process of analyzing and storing blood glucose data within the MCU of the system.

FIG. 1F is a block diagram describing the process for continuous monitoring of the blood glucose levels using trained neural network models.

FIG. 2A is an illustration of the example niCGM device 10 disposed on the skin of the user.

FIG. 2B shows an example console app 1600 which was used to control the niCGM device 10 and receive results of blood glucose measurement.

FIGS. 3A-3E are the accuracy comparison results of the test data from volunteer #11 among Sannuo, Abbott, and the example non-invasive continuous glucose monitor (niCGM) device.

FIGS. 4A-4E are the accuracy comparison results of the test data from volunteer #12 among Sannuo, Abbott and the example niCGM device.

FIGS. 5A-5E are the accuracy comparison results of the test data from volunteer #13 among Sannuo, Abbott, and the example niCGM device.

FIGS. 6A-6E are the accuracy comparison results of the test data from volunteer #14 among Sannuo, Abbott, and the example niCGM device.

FIGS. 7A-7E are the accuracy comparison results of the test data from volunteer #15 among Sannuo, Abbott, and the example niCGM device.

FIGS. 8A-8B are the comparison results of the test data over the 5 volunteers (volunteer #11-15) between Abbott and the example niCGM device.

FIG. 9A is a schematic diagram of an example system 2000 comprising an example niCGM device 2100.

FIG. 9B is a schematic diagram of the printed circuit board assembly PCBA 2200.

FIG. 9C is a flow chart showing the steps of example operations when the embedded software 2300 being executed.

FIG. 9D is a flow chart of the DMLF module 2400 of a data processing system.

FIG. 9E depicts a basis model MLP neural network architecture 2600.

FIG. 9F shows a single layer MLP for the meta model.

FIG. 9G shows a two-layer MLP for the meta model.

FIG. 10A is a schematic diagram of another example blood glucose monitoring system 3000 comprising a device 3100 of another example niCGM and a server 3400.

FIG. 10B is a flow chart showing the steps of example operations when the embedded software 3300 being executed.

FIG. 11A is a schematic diagram of another example blood glucose monitoring system 4000 comprising a device 4100 of another example niCGM, a mobile apparatus 4600 containing console APP 4610 and a remote server 4400 containing DMLF module 4410.

FIG. 11B is a flow chart showing the steps of example operations when the embedded software 4300 being executed.

FIG. 11C is a flow chart showing the steps of example overall operations of the console APP 4610.

### DETAILED DESCRIPTION

### Definitions

Before explaining at least one aspect of the disclosed and/or claimed inventive concept(s) in detail, it is to be understood that the disclosed and/or claimed inventive concept(s) is not limited in its application to the details of construction and the arrangement of the components or steps or methodologies set forth in the following description or illustrated in the drawings. The disclosed and/or claimed inventive concept(s) is capable of other aspects or of being practiced or carried out in various ways. Also, it is to be understood that the phraseology and terminology employed herein is for the purpose of description and should not be regarded as limiting.

As utilized in accordance with the disclosure, the following terms, unless otherwise indicated, shall be understood to have the following meanings.

Unless otherwise defined herein, technical terms used in connection with the disclosed and/or claimed inventive concept(s) shall have the meanings that are commonly understood by those of ordinary skill in the art. Further, unless otherwise required by context, singular terms shall include pluralities and plural terms shall include the singular.

The singular forms “a”, “an”, and “the” include plural forms unless the context clearly dictates otherwise specified or clearly implied to the contrary by the context in which the reference is made. Where a range is referred in the specification, the range is understood to include each discrete point within the range. For example, 1-7 means 1, 2, 3, 4, 5, 6, and 7. The term “Comprising” and “Comprises of” includes the more restrictive claims such as “Consisting essentially of” and “Consisting of”.

For purposes of the following detailed description, other than in any operating examples, or where otherwise indicated, numbers that express, for example, quantities of ingredients used in the specification and claims are to be understood as being modified in all instances by the term “about”. The numerical parameters set forth in the specification and attached claims are approximations that may vary depending upon the desired properties to be obtained in carrying out the invention.

All percentages, parts, proportions, and ratios as used herein, are by weight of the total composition, unless otherwise specified. All such weights as they pertain to listed ingredients are based on the active level and, therefore; do not include solvents or by-products that may be included in commercially available materials, unless otherwise specified.

All publications, articles, papers, patents, patent publications, and other references cited herein are hereby incorporated herein in their entirety for all purposes to the extent consistent with the disclosure herein.

The use of the term “at least one” will be understood to include one as well as any quantity more than one, including but not limited to, 1, 2, 3, 4, 5, 10, 15, 20, 30, 40, 50, 100, etc. The term “at least one” may extend up to 100 or 1000 or more depending on the term to which it is attached. In addition, the quantities of 100/1000 are not to be considered limiting as lower or higher limits may also produce satisfactory results.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

It shall be understood that for every embodiment in which the term “comprising” (or any related form such as “comprise” and “comprises”), “including” (or any related forms such as “include” or “includes”), or “containing” (or any related forms such as “contain” or “contains”) is used, this disclosure/application also includes alternate embodiments where the term “comprising”, “including,” or “containing.” is replaced with “consisting essentially of” or “consisting of”. These alternate embodiments that use “consisting of” or “consisting essentially of” are understood to be narrower embodiments of the “comprising”, “including,” or “containing,” embodiments.

For the sake of clarity, “comprising”, “including”, “containing” and “having”, and any related forms are open-ended terms which allows for additional elements or features beyond the named essential elements, whereas “consisting of” is a closed end term that is limited to the elements recited in the claim and excludes any element, step, or ingredient not specified in the claim.

“Consisting essentially of” limits the scope of a claim to the specified materials, components, or steps (“essential elements”) that do not materially affect the essential characteristic(s) of the claimed invention. In some embodiments, the essential characteristics are the basic and novel characteristic(s) of the claimed invention.

For the sake of clarity, “characterized by” or “characterized in” (together with their related forms as described above), does not limit or change the nature of whether the list of terms following it are open or closed. For example, in a claim directed towards “a device comprising A, B, C, and characterized by D, E, and F”, the elements D, E, and F are still open-ended terms and the claim is meant to include other elements due to the use of the word “comprising” earlier in the claim.

The term “each independently selected from the group consisting of” means when a group appears more than once in a structure, that group may be selected independently each time it appears.

The term “artificial neural network” refers to a computational architecture having programmed instructions that is capable of learning from a training data set to make one or more predictions such as predictions of properties of new test objects.

The term “computer system” refers to an electronic device that includes a memory configured to store coded instructions, a processor to execute the instructions, an output interface, etc., capable of performing various claimed steps of the present invention.

In a non-limiting embodiment, the present application discloses a wearable or otherwise portable device, such as an arm band or watch, that is configured to provide continuous and noninvasive monitoring of a wearer's blood glucose levels (BGL). In one embodiment, the device utilizes a near-infrared (NIR) light emitting diode (LED) sensor and machine learning algorithms to accurately predict and continuously provide a wearer's BGL in real time.

As used herein, the term “about” is understood as within a range of normal tolerance in the art and not more than +20% of a stated value. By way of example only, about 940 means from 752 to 1128 including all values in between. As used herein, the phrase “about” a specific value also includes the specific value, for example, about 940 includes 940. For example, the values of the wavelengths described herein include the peak value and ±20% bandwidth coverage.

As used herein and in the claims, the terms “general” or “generally”, or “substantial” or “substantially” mean that the recited characteristic, angle, shape, state, structure, or value need not be achieved exactly, but that deviations or variations, including for example, tolerances, measurement error, measurement accuracy limitations and other factors known to those of skill in the art, may occur in amounts that do not preclude the effect the characteristic was intended to provide. For example, an object that has a “generally” cylindrical shape would mean that the object has either an exact cylindrical shape or a nearly exact cylindrical shape. In another example, an object that is “substantially” perpendicular to a surface would mean that the object is either exactly perpendicular to the surface or nearly exactly perpendicular to the surface, e.g., has a 5% deviation.

The term “light emitter” refers to an element or a component that can emit light. For example, a light emitter is or comprises one or more “light transmitters” or a “transmitters” such as light-emitting diodes (LEDs). For example, a light emitter is or comprises one or more near infrared light emitting diodes. For example, a light emitter is or comprises a NIR transceiver with LEDs having nominal wavelengths of about 940 nm, 1350 nm, and/or 1500 nm with the bandwidth of ±20%.

The term “light receiver” refers to an element or a component that can receive light, or light signals. In some examples, a light receiver is or comprises a photodetector or infrared receiver chip that can receive light signals from the transmitters such as LEDs.

The term “controller” refers to an element or a component that controls the operation of the components or elements in a device.

The term “blood glucose monitoring” is a process of measuring the blood glucose level of a user over a period of time. For example, the blood glucose levels can be measured regularly. The term “device for blood glucose monitoring” means a device that can perform a single measurement of the blood glucose level of a user at a specific time point, as well as multiple measurements of the blood glucose level of a user over a period of time, for example, regularly or continuously. The term “continuous glucose monitoring (CGM)” is a process of measuring the blood glucose level of a user continuously.

The terms “light”, “light wave” and “light signals” are interchangeable and refer to a form of electromagnetic radiation emitted by the light emitter at a certain wavelength. In certain embodiments, the light can be emitted within a defined time period as a light pulse. For example, the light can be one or more near infrared (NIR) light having one or more defined wavelengths, for example, about 940 nm, about 1350 nm, and/or about 1500 nm.

The terms “reflected light”, “reflected light signal” or “reflected light wave” are interchangeable and refer to the light reflected from the target surface of a user. For example, the light may penetrate the epidermis, encounter glucose molecules within the user's blood vessels, and get reflected back to the light receiver.

The term “data vector” refers to a numerical (digital) representation of a data point or a set of data points generated from the reflected light signal group, used as input to a machine learning model. In some examples, the data point or a set of data points are converted from a light signal or a light signal group. The term “data vector stream” refers to a continuous or regular flow of multiple data vectors within a period of time.

The terms “deep meta-learning framework”, “meta deep learning framework” and “DMLF” are used interchangeably herein to describe a framework of machine learning algorithms that involve training multiple artificial neural networks for learning from other learning algorithms.

Unless specifically stated otherwise, it is appreciated that throughout this specification discussions utilizing the terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

Implementations of the methods disclosed herein may be performed in the operation of such computing devices. The order of the blocks presented in the examples above can be varied for example, blocks can be re-ordered, combined, and/or broken into sub-blocks. Certain blocks or processes can be performed in parallel.

The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or value beyond those recited. Headings, lists, and numbering included herein are for ease of explanation only and are not meant to be limiting.

It will also be understood that, although the terms “first,” “second,” etc. may be used herein to describe various elements, these elements should not be limited by these terms. These terms are only used to distinguish one element from another. For example, a first node could be termed a second node, and, similarly, a second node could be termed a first node, which changing the meaning of the description, so long as all occurrences of the “first node” are renamed consistently and all occurrences of the “second node” are renamed consistently. The first node and the second node are both nodes, but they are not the same node.

It is to be understood that terms such as “top”, “bottom”, “middle”, “side” “length”, “inner”, “outer”, “interior”, “exterior”, “outside”, “vertical”, “horizontal” and the like as may be used herein, merely describe points of reference and do not limit the present invention to any particular orientation or configuration.

As used herein and in the claims, “couple” or “connect” refers to electrical coupling or connection either directly or indirectly via one or more electrical means unless otherwise stated.

As used herein and in the claims, the term “back-to-back sequentially” refers to steps, processes, or events to occur one after the other in a specific order or sequence in one or more repeated cycles. In some embodiments, the transmitter is controlled to emit the first light signal at a first time interval, then emit the second light signal at a second time interval, and finally the third light signal at a first time interval in the first cycle. After the first cycle, the steps are repeated in the second cycle and so forth.

The terminology used herein is for the purpose of describing particular implementations only and is not intended to be limiting of the claims. As used in the description of the implementations and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined that a stated condition precedent is true]” or “if a stated condition precedent is true]” or “when a stated condition precedent is true]” may be construed to mean “upon determining” or “in response to determining” or “in accordance with a determination” or “upon detecting” or “in response to detecting” that the stated condition precedent is true, depending on the context.

The foregoing description and summary of the invention are to be understood as being in every respect illustrative and exemplary, but not restrictive, and the scope of the invention disclosed herein is not to be determined only from the detailed description of illustrative implementations but according to the full breadth permitted by patent laws. It is to be understood that the implementations shown and described herein are only illustrative of the principles of the present invention and that various modifications may be implemented by those skilled in the art without departing from the scope and spirit of the invention.

Although the description referred to particular embodiments, the disclosure should not be construed as limited to the embodiments set forth herein.

### EXAMPLES

Provided herein are examples that describe in more detail certain embodiments of the present disclosure. The examples provided herein are merely for illustrative purposes and are not meant to limit the scope of the invention in any way. All references given below and elsewhere in the present application are hereby included by reference.

### Example 1A

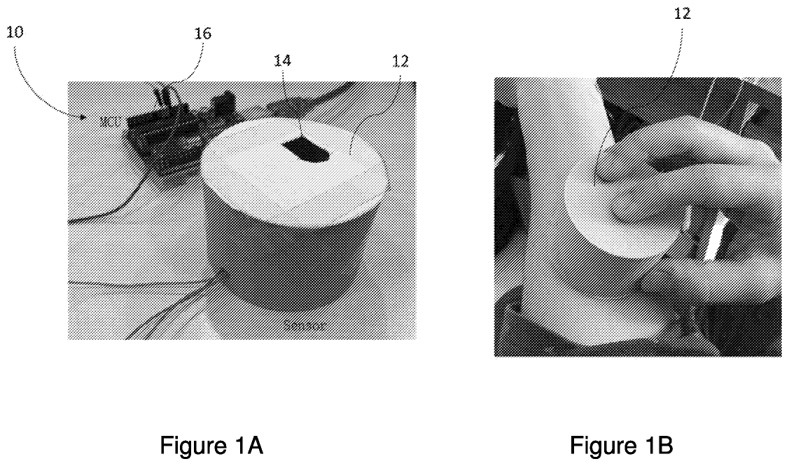

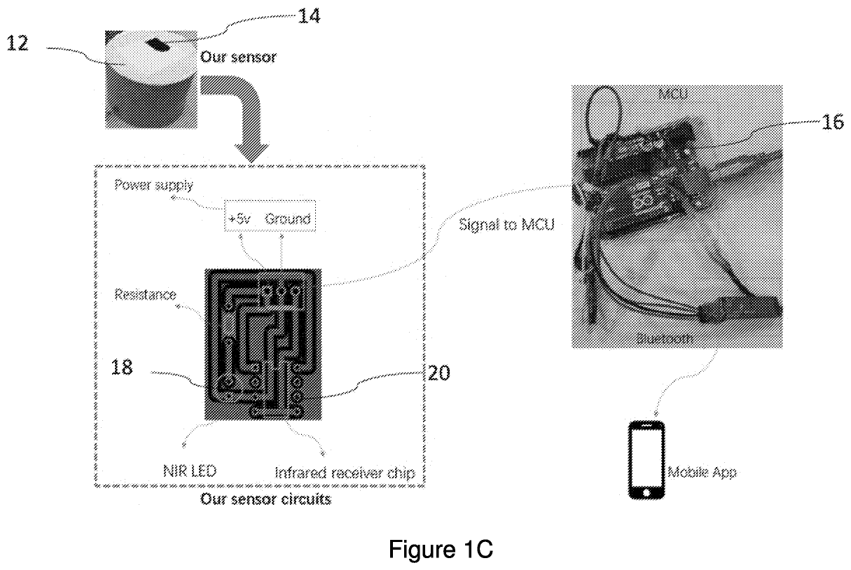

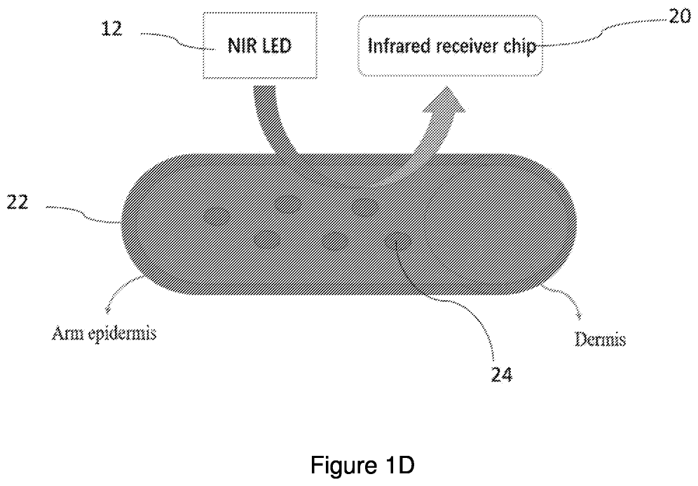

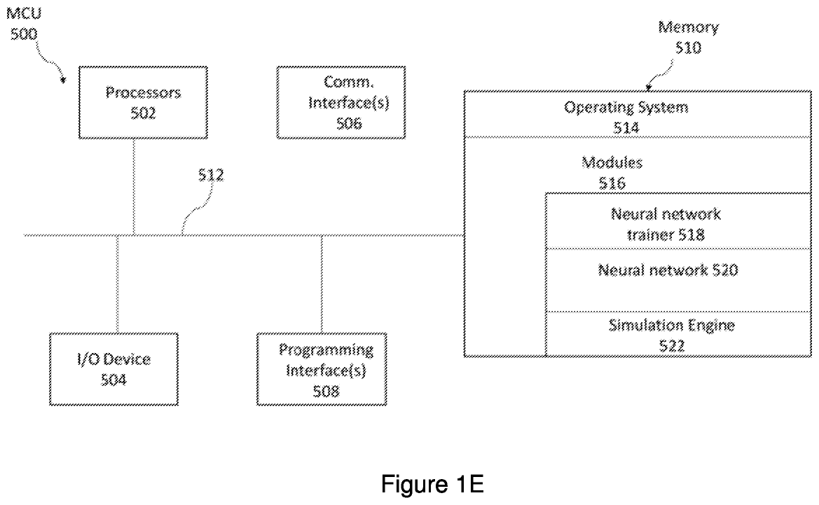

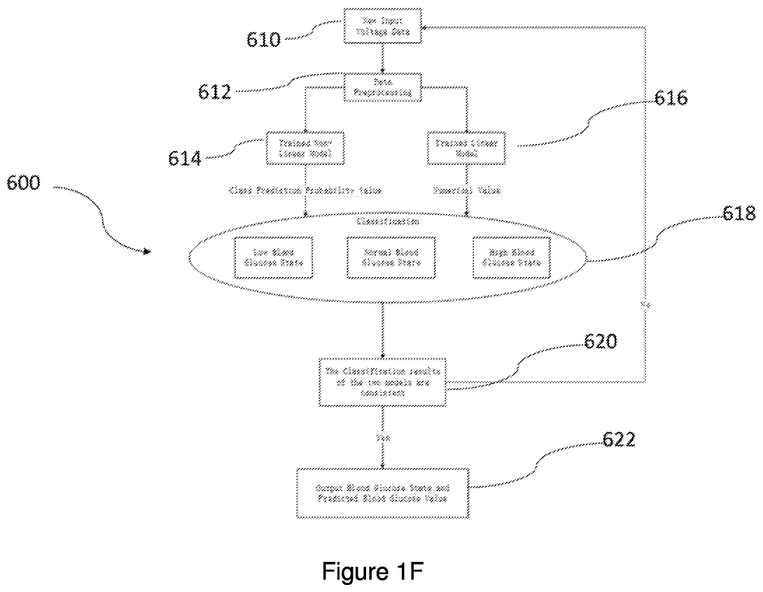

FIG. 1A is a perspective view of one embodiment of the portable device 10, including a housing body 12 and a sensor 14 disposed within the housing body 12. As shown in FIG. 1A, the sensor 14 may be connected to a microcontroller unit (MCU) 16. In another embodiment (not shown), the MCU 16 is disposed within the housing body 12. In use, as shown in FIG. 1B, the portable device 10 may be disposed on the skin of the user, in one embodiment, on the inside surface of the user's forearm, with the sensor 14 positioned over a user's arteries, blood vessels, capillaries, etc., so that it may obtain readings from the user's blood.

As shown in FIG. 1C, the optical sensor 14 includes, among other components, a circuit with a near infrared light emitting diode 18 and an infrared receiver chip 20. In one embodiment, the NIR LED 18 is configured to emit an infrared signal within the near infrared region of the electromagnetic spectrum (from 780 nm to 2500 nm). In one embodiment, the wavelength of the NIR LED 18 is about 940 nm. A commercially available NIR LED may be obtained from China Young Sun LED Technology Company. Ltd. The optical sensor also includes an infrared receiver chip 20 that is configured to receive the signal reflected from the user's arm (as shown in FIG. 1D). In one embodiment, the infrared receiver chip 20 is a monolithic photodiode with an on-chip transimpedance amplifier, such as OPT101 from Texas Instruments.

As shown in FIG. 1D, the infrared signal from the NIR LED 18 is directed toward the body of the user, in one example the inside surface of the user's forearm, where it penetrates the epidermis 22, encountering glucose molecules 24 within the user's blood vessels, and is reflected back to the infrared receiver chip 20.

Referring again to FIG. 1C, the infrared receiver chip 20 converts the reflected infrared light signal into an analog voltage signal, which is then transmitted to a built-in high-precision analog-to-digital converter (ADC) within the MCU 16 to obtain the digital voltage signal, x. That digital voltage signal, x, is then transmitted to a trained neural network model, that yields a predicted BGL. The predicted BGL may then be transmitted via Bluetooth, or other suitable protocol, to an application stored on the user's phone or computer or a cloud server.

FIG. 1E is a block diagram of an example system architecture of an exemplary device (or MCU) configured to train, store, and/or use a neural network in accordance with one or more implementations. While certain specific features are illustrated, those skilled in the art will appreciate from the present disclosure that various other features have not been illustrated for the sake of brevity, and so as not to obscure more pertinent aspects of the implementations disclosed herein. To that end, as a non-limiting example, in some implementations the device 100 includes one or more processing units 102 (e.g., microprocessors, ASICs, FPGAs, GPUs, CPUs, processing cores, or the like), one or more input/output (I/O)) devices 104, one or more communication interfaces 106 (e.g., USB, IEEE 802.3x, IEEE 802.11x, IEEE 802.16x, GSM, CDMA, TDMA, GPS, IR, BLUETOOTH, ZIGBEE, SPI, I2C, or the like type interface), one or more programming (e.g., I/O) interfaces 108, a memory 110, and one or more communication buses 112 for interconnecting these and various other components. In some implementations, the one or more communication buses 112 include circuitry that interconnects and controls communications between system components.

The memory 110 includes high-speed random-access memory, such as DRAM, SRAM, DDR RAM, or other random-access solid-state memory devices. In some implementations, the memory 110 includes non-volatile memory, such as one or more magnetic disk storage devices, optical disk storage devices, flash memory devices, or other non-volatile solid-state storage devices. The memory 110 optionally includes one or more storage devices remotely located from the one or more processing units 102. The memory 110 comprises a non-transitory computer readable storage medium. In some implementations, the memory 110 or the non-transitory computer readable storage medium of the memory 110 stores the following programs, modules and data structures, or a subset thereof including an optional operating system 114 and one or more modules 116. The operating system 114 includes procedures for handling various basic system services and for performing hardware dependent tasks. The neural network trainer 118 is an example of a module that can be configured to train a neural network 120 according to the techniques disclosed herein. The neural network 120 represents a neural network that has been integrated into an application or otherwise trained and then stored in the memory 110. In one embodiment, the neural networks 120 may include both linear and non-linear models. The simulation engine 122 is an example of a module that can be configured to predict the BGL of a user, as described herein.

In some implementations, the neural network 120 is a type of machine learning model. Alternatively, other machine learning models may be utilized other than a neural network (e.g., an artificial neural network), such as a decision tree, support vector machine, Bayesian network, or the like, in order to predict a user's blood glucose level in real time based on the obtained digital voltage signals. In some implementations, the neural network trainer 118 trains the neural network 120 to predict a user's blood glucose level based on aggregating and classifying multiple user's blood glucose levels using statistical and/or machine learning techniques based on the variability of the acquired digital voltage signals. In some implementations, the blood glucose data is classified based on comparing the variability of the digital voltage signals to a threshold. For example, the techniques described herein could classify a user's obtained digital voltage signal to determine a blood glucose state as either a low blood glucose state, a normal blood glucose state, and/or a high blood glucose state.

FIG. 1E is intended more as a functional description of the various features which are present in a particular implementation as opposed to a structural schematic of the implementations described herein. As recognized by those of ordinary skill in the art, items shown separately could be combined and some items could be separated. The actual number of units and the division of particular functions and how features are allocated among them will vary from one implementation to another and, in some implementations, depends in part on the particular combination of hardware, software, or firmware chosen for a particular implementation.

FIG. 1F is a flow chart illustrating an exemplary method 100 of predicting blood glucose levels using trained neural network models. In some implementations, the method 100 is performed by a device (MCU) (e.g., device 100 of FIG. 1E). The method 100 can be performed at a mobile device, desktop, laptop, or server device. In some implementations, the method 100 is performed by processing logic, including hardware, firmware, software, or a combination thereof. In some implementations, the method 100 is performed by a processor executing code stored in a non-transitory computer-readable medium (e.g., a memory).

At block 110, the method obtains new input digital voltage data, x, from the ADC converting the analog voltage data received from the infrared receiver chip, and transmits that data to be processed 112. The data, x, is then simultaneously subjected to a trained non-linear model 114 and a linear model 116, which produce class prediction probability values and numerical values, respectively. The data is then classified 118 as being low, normal, or high and the classification results of the two models are compared 120 to determine if the values are consistent.

If so, the output blood glucose state and predicted blood glucose values are determined 122.

In one example, the nonlinear model produces a probability for each of the three glucose states, i.e. low (0.2), normal (0.6), and high glucose levels (0.2). The state with the highest probability is considered the true state. In this example, the normal glucose state would be considered the true state. Then, the linear model is configured to produce a numerical value for the glucose level, for example 5.9. According to the pre-defined intervals for the three states (e.g., 0-4 for low, 4-8 for normal, and >8 for high), the linear model will also result in one of the three states. In this example, 5.9 would be considered to be normal. Therefore, the nonlinear and linear models produce consistent results.

The machine learning models 114 and 116 are trained to predict the BGL of a user based on digital infrared light reflection signals x, converted from analog infrared light reflection signals collected by the infrared light receiving chip 20. The machine learning models are trained by comparing a predicted BGL based on the voltage signal x with an experimentally-determined BGL obtained through a professionally certified invasive blood glucose device.

The linear model 116 may utilize the polynomial regression model. For example, one example the non-linear mode is an ANN (Artificial Neural Network) model. It should be understood that other models, such as a convolutional neural network (CNN), recurrent neural network (RNN), long short-term memory (LSTM), or generative adversarial network (GAN) may also be used.

During an iterative training process, the machine learning models may make predictions based on such input, compare those output predictions with experimentally-determined BGL and adjust the configuration of the machine learning model to reduce differences between predictions and experimentally-known results in future iterations. Training such a model using a number of collected BGLs can provide a machine learning model that is accurate with respect to predicting the BGL of the user.

### Example 1B

### Example Device and System Architecture

In this example, the example glucose monitoring portable device 10 as described in Example 1A including a light emitter with a NIR LED 18 having a wavelength of about 940 nm, a light receiver (which is the infrared receiver chip 20), a controller (which is the MCU 16) and an enclosure (which is the housing body 12) was used for experiment. The example device 10 produces an infrared signal, emitted by the NIR LED 18, which is directed towards the surface of a user's forearm, where it penetrates into the epidermis, encountering blood glucose molecules within the user's blood vessels, and is reflected back to the light receiver (infrared receiver chip 20 in this example). Then, the reflected infrared light signals were converted into an analog voltage signal (digital signal) in the MCU 16, which, after preprocessing, is then transmitted to a data processing system having a trained machine learning module (which is the trained neural network model) as described in Example 1, that eventually yields a predicted blood glucose level of the user. For each measurement, the blood glucose level data were captured for about 2 minutes and a stable value was selected and recorded. In other examples, the blood glucose level data were captured for 1-3 minutes.

### Accuracy Analysis

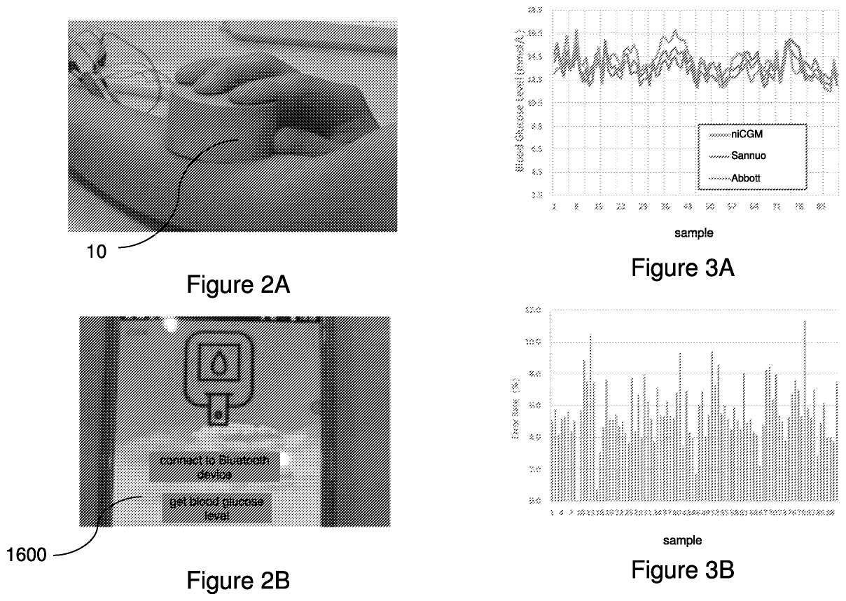

15 volunteers (10 volunteers for data collection for training of the neural network model and 5 volunteers for system performance evaluation) were recruited to evaluate the accuracy of the example non-invasive continuous glucose monitor device (abbreviated as ‘niCGM’ hereinafter) and compare it with the performance of a commercially available device, Abbott's FreeStyle Libre 2 system (abbreviated as ‘Abbott’ hereinafter), which takes a micro-invasive approach and uses a sensor pad to penetrate skin. Sinocare invasive continuous glucose monitor, Blood Glucose Meter (Safe-Accu), (Changsha Sinocare Inc.) (also referred as ‘Sannuo’ hereinafter) was used as the benchmark (as accuracy standard), which pricks a finger using a needle to collect blood and gets the glucose level through a testing paper. FIG. 2A is an illustration of the example niCGM device 10 disposed on the skin of the user. FIG. 2B shows an example console APP 1600 which was used to control the niCGM device 10 and receive results of blood glucose measurement.

The blood glucose levels of the 15 volunteers were collected 3-4 times per day for 30 days with niCGM, Abbitt and Sannuo at the same time, respectively. 10 volunteers' data over 30 days were used to train our deep learning framework, and the rest 5 volunteer's data (volunteers #11-15) were used for testing. The procedures of conducting the blood glucose level determination for Abbott and Sannuo were conducted according to the manufacturers' instructions respectively.

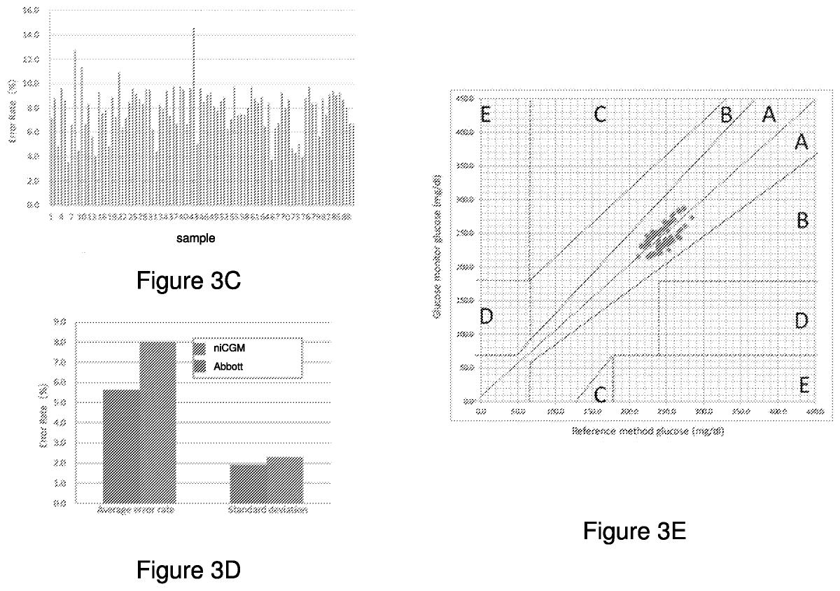

Now referring to FIGS. 3A-3E, the accuracy comparison results of the test data from volunteer #11 among Sannuo, Abbott, and the example device niCGM were shown. The test data were measured from volunteer #11 3 times per day for 30 days to obtain 90 output data points in total, respectively.

The glucose level accuracy comparison using the test data from volunteer #11 measured 3 times per day for 30 days is shown in FIG. 3A. The results showed that the overall blood glucose levels of the example device niCGM high correlation with that of Sannuo, the benchmark. The error rates of the example device niCGM from the test data in FIG. 3A were shown in FIG. 3B. Results showed that about 97.78% of the error rates are below 10%, and 100% of the error rates are below 15%, indicating that the example device in the present disclosure has extremely low error rates throughout the 90 days testing period. The error rates of Abbott's glucose monitor from the test data in FIG. 3A were shown in FIG. 3C. Results showed that 95.56% of the error rates are below 10%, and 100% of the error rates are below 15%, indicating that the example device in the present disclosure has comparable or even lower error rates throughout the 90 days testing period when compared to micro-invasive device Abbott. As shown in FIG. 3D, the average error rate and standard deviation (SD) of the error rates of the example device niCGM were 5.6 and 1.9, respectively, while the average error rate and standard deviation of the error rates of Abbott's glucose monitor are 8.0 and 2.3, respectively, indicating that the example device in the present disclosure has significantly lower error rate and standard deviation of the error rates when compared to that of Abbott. Now referring to FIG. 3E, a chart showing the Clark error grid analysis of the example device niCGM with Sannuo as the reference sensor. Region A are those values within 20% of the reference sensor. Region B contains points that are outside of 20% but would not lead to inappropriate treatment. Region C are those points leading to unnecessary treatment. Region D are those points indicating a potentially dangerous failure to detect hypoglycemia or hyperglycemia. Region E are those points that would confuse treatment of hypoglycemia for hyperglycemia and vice versa. Results showed that the example device niCGM achieved 100% accuracy in Zone A (i.e., values within 20% of the reference sensor) of CEG (Consensus Error Grid) system as shown in FIG. 3E.

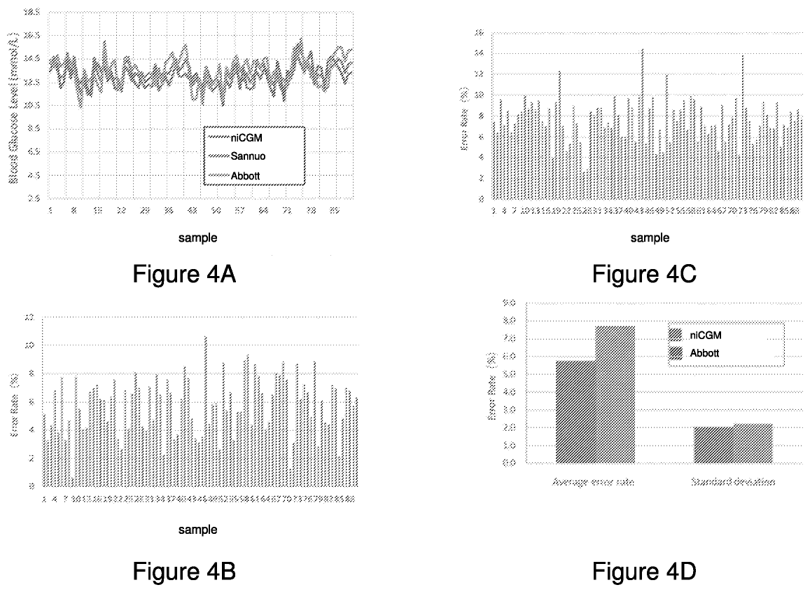

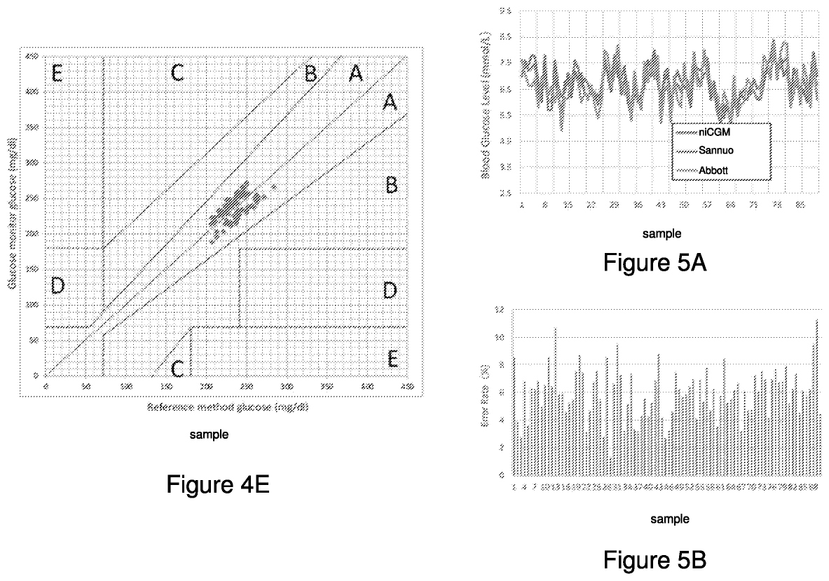

Now referring to FIGS. 4A-4E, the accuracy comparison results of the test data from volunteer #12 among Sannuo, Abbott and the example device niCGM were shown. Similarly, the test data were measured from volunteer #12 3 times per day for 30 days to obtain 90 output data points in total, respectively.

The glucose level accuracy comparison using the test data from volunteer #12 measured 3 times per day for 30 days were shown in FIG. 4A. The results showed that the overall blood glucose levels of the example device niCGM high correlation with that of Sannuo, the benchmark. The error rates of the example device niCGM from the test data in FIG. 4A were shown in FIG. 4B. Results showed that 98.89% of the error rates were below 10%, and 100% of the error rates were below 15%, indicating that the example device in the present disclosure has extremely low error rates throughout the 90 days testing period. The error rates of Abbott's glucose monitor from the test data in FIG. 4A were shown in FIG. 4C. Results showed that 95.56% of the error rates were below 10%, and 100% of the error rates were below 15%, indicating that the example device in the present disclosure has comparable or even lower error rates throughout the 90 days testing period when compared to micro-invasive device Abbott. As shown in FIG. 4D, the average error rate and standard deviation of the error rates of our glucose monitor are 5.7 and 2.0, respectively, while the average error rate and standard deviation of the error rates of Abbott were 7.7 and 2.2, respectively, indicating that the example device in the present disclosure has significantly lower error rate and standard deviation of the error rates when compared to that of Abbott. Now referring to FIG. 4E, a chart showing the Clark error grid analysis of niCGM with Sannuo as the reference sensor using the test data from volunteer #12 measured 3 times per day for 30 days. Results also showed that the example device niCGM achieved 100% accuracy in Zone A (i.e., values within 20% of the reference sensor) of CEG (Consensus Error Grid) system as shown in FIG. 4E.

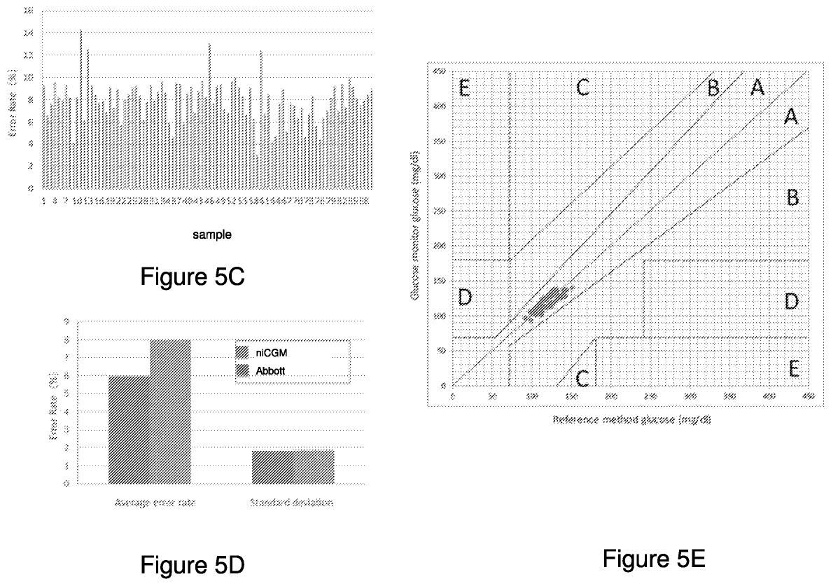

Now referring to FIGS. 5A-5E, the accuracy comparison results of the test data from volunteer #13 among Sannuo, Abbott, and the example device niCGM were shown. Similarly, the test data were measured from volunteer #13 3 times per day for 30 days to obtain 90 output data points in total, respectively.

The glucose level accuracy comparison using the test data from volunteer #13 measured 3 times per day for 30 days is presented in FIG. 5A. The overall blood glucose levels of the example device niCGM high correlation with that of Sannuo, the benchmark. The error rates of the example device niCGM from the test data in FIG. 5A were shown in FIG. 5B. Results showed that 97.78% of the error rates were below 10%, and 100% of the error rates were below 15%, indicating that the example device in the present disclosure has extremely low error rates throughout the 90 days testing period. The error rates of Abbott from the test data in FIG. 5A were shown in FIG. 5C. Results showed that 95.56% of the error rates were below 10%, and 100% of the error rates were below 15%, indicating that the example device in the present disclosure has comparable or even lower error rates throughout the 90 days testing period when compared to micro-invasive device Abbott. As shown in FIG. 5D, the average error rate and standard deviation of the error rates of the example device niCGM were 5.85 and 2.03, respectively, while the average error rate and standard deviation of the error rates of Abbott are 7.99 and 2.01, respectively, indicating that the example device in the present disclosure has significantly lower error rate and standard deviation of the error rates when compared to that of Abbott. Now referring to FIG. 5E, a chart showing the Clark error grid analysis of the example device niCGM with Sannuo as the reference sensor using the test data from volunteer #13 measured 3 times per day for 30 days. Results showed that the example device niCGM achieved 100% accuracy in Zone A (i.e., values within 20% of the reference sensor) of CEG (Consensus Error Grid) system as shown in FIG. 5E.

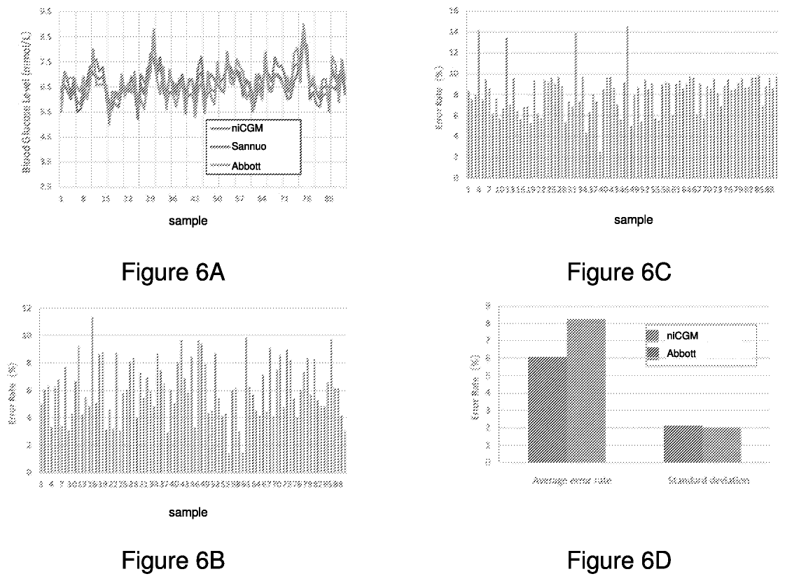

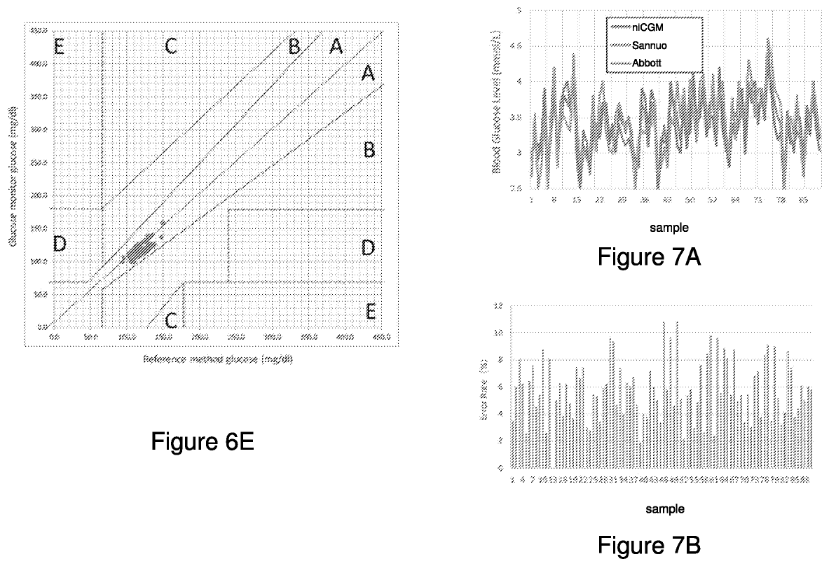

Now referring to FIGS. 6A-6E, the accuracy comparison results of the test data from volunteer #14 among Sannuo, Abbott, and the example device niCGM were shown. Similarly, the test data were measured from volunteer #14 3 times per day for 30 days to obtain 90 output data points in total, respectively.

The glucose level accuracy comparison using the test data from volunteer #14 measured 3 times per day for 30 days was presented in FIG. 6A. The overall blood glucose levels of the example device niCGM high correlation with that of Sannuo, the benchmark. The error rates of the example device niCGM from the test data in FIG. 6A were shown in FIG. 5B. Results showed that 98.89% of the error rates were below 10%, and 100% of the error rates were below 15%, indicating that the example device in the present disclosure has extremely low error rates throughout the 90 days testing period. The error rates of Abbott from the test data in FIG. 6A were shown in FIG. 6C. Results showed that 95.56% of the error rates were below 10%, and 100% of the error rates were below 15%, indicating that the example device in the present disclosure has comparable or even lower error rates throughout the 90 days testing period when compared to micro-invasive device Abbott. As shown in FIG. 5D, the average error rate and standard deviation of the error rates of the example device niCGM were 6.07 and 2.11, respectively, while the average error rate and standard deviation of the error rates of Abbott were 8.23 and 2.00 respectively, indicating that the example device in the present disclosure has significantly lower error rate and standard deviation of the error rates when compared to that of Abbott. Now referring to FIG. 6E, a chart showing the Clark error grid analysis of the example device niCGM with Sannuo as the reference sensor using the test data from volunteer #14 measured 3 times per day for 30 days. Results showed that the example device niCGM achieved 100% accuracy in Zone A (i.e., values within 20% of the reference sensor) of CEG (Consensus Error Grid) system as shown in FIG. 6E.

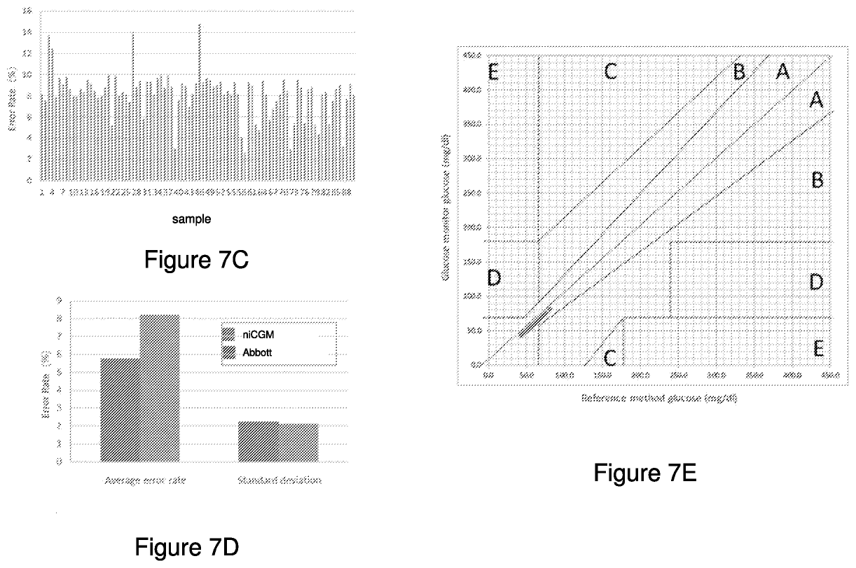

Now referring to FIGS. 7A-7E, the accuracy comparison results of the test data from volunteer #15 among Sannuo, Abbott, and the example device niCGM were shown. Similarly, the test data were measured from volunteer #15 3 times per day for 30 days to obtain 90 output data points in total, respectively.

The glucose level accuracy comparison using the test data from volunteer #15 measured 3 times per day for 30 days was presented in FIG. 7A. The overall blood glucose levels of the example device niCGM high correlation with that of Sannuo, the benchmark. The error rates of the example device niCGM from the test data in FIG. 7A were shown in FIG. 7B. Results showed that 98.89% of the error rates were below 10%, and 100% of the error rates were below 15%, indicating that the example device in the present disclosure has extremely low error rates throughout the 90 days testing period. The error rates of Abbott from the test data in FIG. 7A is shown in FIG. 7C. Results showed that 95.56% of the error rates were below 10%, and 100% of the error rates were below 15%, indicating that the example device in the present disclosure has comparable or even lower error rates throughout the 90 days testing period when compared to micro-invasive device Abbott. As shown in FIG. 7D, the average error rate and standard deviation of the error rates of the example device niCGM were 5.77 and 2.25, respectively, while the average error rate and standard deviation of the error rates of Abbott were 8.21 and 2.11, respectively, indicating that the example device in the present disclosure has similar or even lower error rate and standard deviation of the error rates when compared to that of Abbott. Now referring to FIG. 7E, a chart showing the Clark error grid analysis of our niCGM with Sannuo CGM as the reference sensor using the test data from volunteer #12 measured 3 times per day for 30 days. Results showed that the example device niCGM achieved 100% accuracy in Zone A (i.e., values within 20% of the reference sensor) of CEG (Consensus Error Grid) system as shown in FIG. 7E.

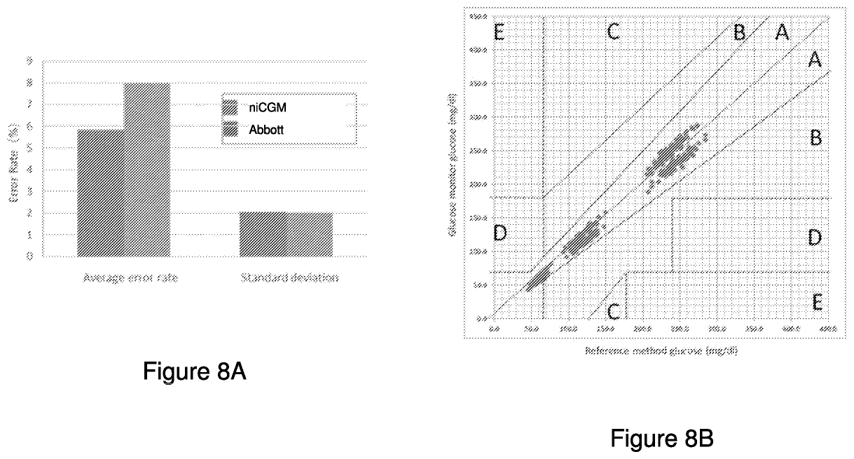

Now referring to FIGS. 8A-8B, the comparison results of the preceding test data over the 5 volunteers (volunteer #11-15) between Abbott and the example device niCGM were shown. From the results presented above, it clearly showed that the example device niCGM and system thereof achieved better accuracy than Abbott's micro-invasive FreeStyle Libre 2. Specifically, the average error rate and standard deviation of the error rates of the example device niCGM for volunteers #11, #12, #13, #14, and #15 were 5.83 and 2.03, respectively, while the average error rate and standard deviation of the error rates of Abbott's FreeStyle Libre 2 for volunteers #11, #12, #13, #14, and #15 were 7.99 and 2.01, respectively. The comparison results were summarized in FIG. 8A. Now referring to FIG. 8B, a chart showing the Clark error grid analysis of the example device niCGM with Sannuo as the reference sensor using the test data from volunteers #11-15. The example device niCGM achieved 100% accuracy in Zone A of CEG (Consensus Error Grid) system as shown in FIG. 8B.

In summary, the results showed that the outstanding performance regarding the accuracy of the example non-invasive continuous glucose monitoring device (niCGM) and system thereof when compared to that of Abbott's micro-invasive glucose monitor FreeStyle Libre 2 using the performance of Sannuo's invasive glucose monitor as a benchmark reference sensor. The results showed that the example device and system niCGM's average error rate and the standard deviation of the error rates were 5.83 and 2.03, respectively, while those of Abbott's FreeStyle Libre 2 were 7.99 and 2.01, respectively, indicating that the example device in the present disclosure has significantly lower error rate and standard deviation of the error rates when compared to that of Abbott. Moreover, the example device and system niCGM achieved 100% accuracy in Zone A of CEG (Consensus Error Grid) system.

### Example 2

### Example Blood Glucose Monitoring System and Device

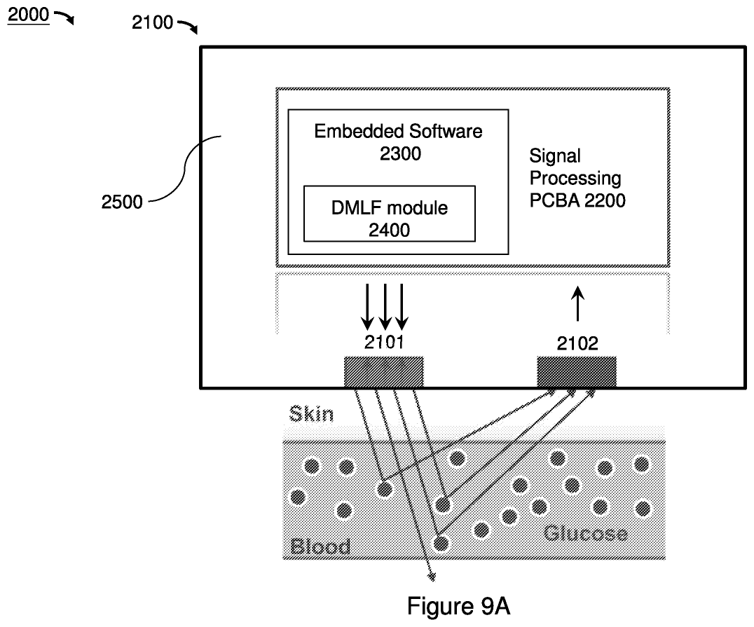

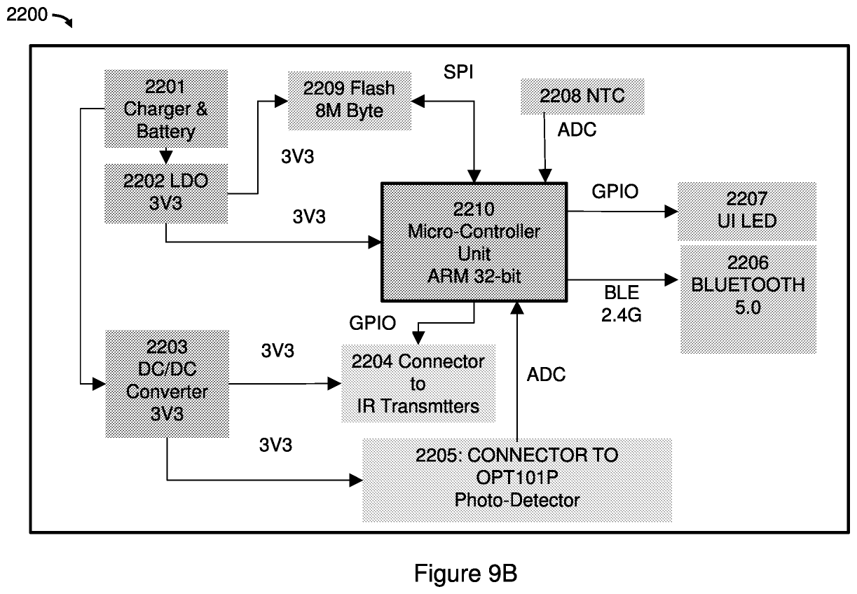

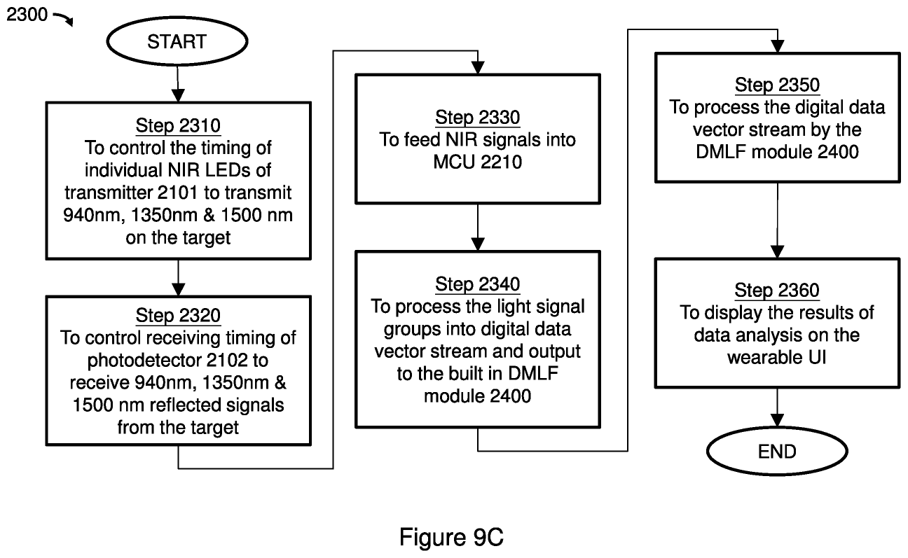

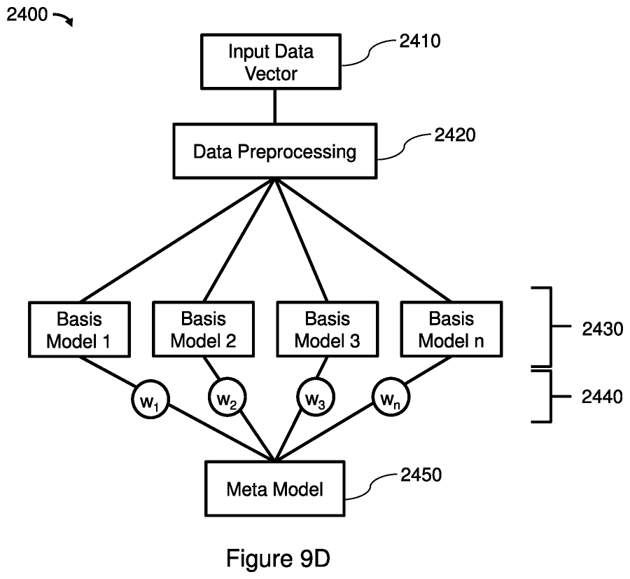

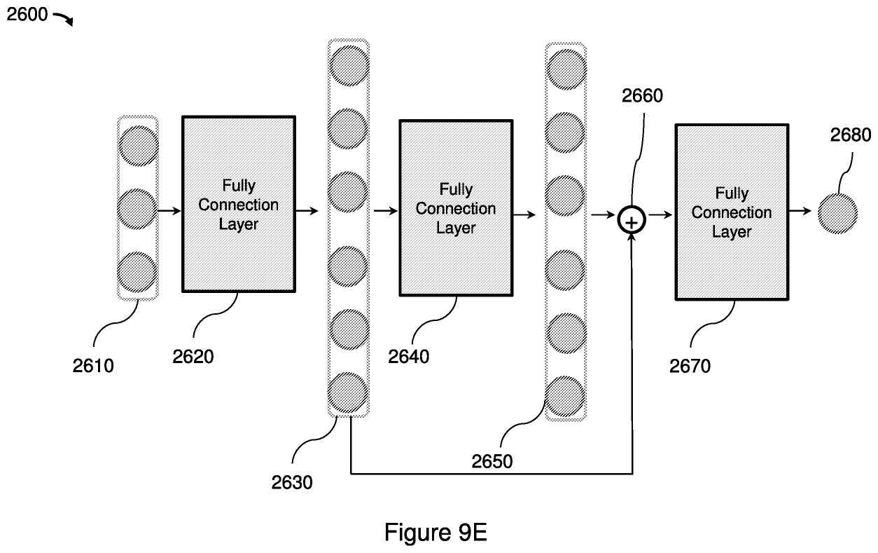

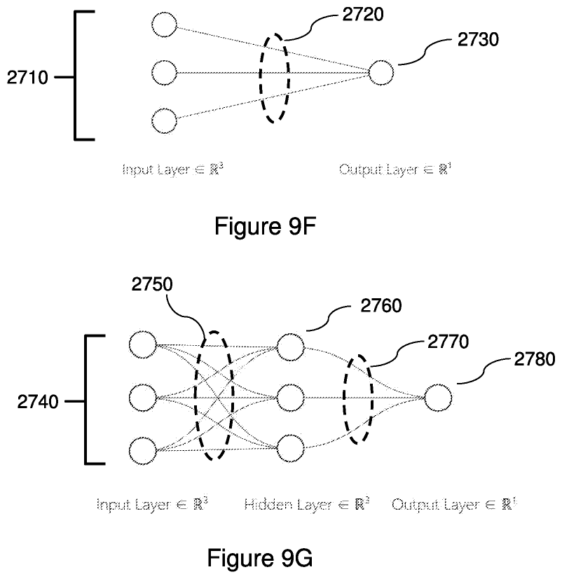

Now referring to FIG. 9A, a schematic diagram of an example system 2000 comprising an example non-invasive continuous glucose monitor (niCGM) device 2100 is shown. In this example, the device 2100 is a wearable device. The device 2100 generally includes an enclosure, a light emitter, a light receiver, and a controller. In this example, the enclosure is or contains a housing body 2500 to house the other components, the light emitter is or contains a transmitter 2101, the light receiver is or contains a photodetector 2102, and the controller is or contains a signal processing, PCBA (printed circuit board assembly) 2200. The transmitter 2101, the photodetector 2102, the PCBA are disposed within the housing body 2500. The PCBA 2200 includes a processor unit coupled with a memory that stores an executable, software program. In this example, the processor unit is a microprocessor and the software program can be named as an embedded software 2300. In this example, the embedded software 2300 further includes an operating module that controls the operation of the device 2100 and a data processing system that processes the data signal, wherein the data processing system includes a machine learning module that analyzes the data signal to generate an output data that is a blood glucose level of the user. In this example, the machine learning module comprises a deep meta-learning framework (DMLF) module 2400 which is a meta learning software plug-in for the device 2100, and will be shown in FIG. 9A and described in more details later. In this example, the transmitter 2101 and the photodetector 2102 are disposed adjacent to each other at a defined distance proximate to the lower surface of the housing body 2500. The transmitter 2101 is configured to emit a first light signal, a second light signal and a third light signal directed to a target surface (e.g., skin) of the user, such that a first reflected light signal, a second reflected light signal and a third reflected light signal (collectively can be named as reflected light signals or reflected light signal groups) that are reflected from the target surface (skin), respectively, can be substantially received by the photodetector 2102.

In some examples, the light emitting portion of the transmitter and/or the signal receiving portion of the photodetector are positioned towards each other at a defined angle such that most or substantially all reflected light signals can be received by the photodetector 2102. In this example, the transmitter 2101 and the photodetector 2102 are positioned at the lower side of the housing body 2500, such that the transmitter 2101 and photodetector 2102 are proximate to the target detection area (such as skin of the forearm or wrist) of a subject or a user.

In some examples, the device 2100 further includes one or more optical refractive lens components to guide the NIR light paths to the PCBA 2200.

The transmitter 2101 and photodetector 2102 are in electrical communication with the PCBA 2200. In other words, PCBA 2200 is operatively connect with the transmitter 2101 and the photodetector 2102. One of the functions of the PCBA 2200 is to control the transmission of lights of transmitters 2101 and capture the response of the reflected light signals of photodetector 2102.

### Example NIR LED Transmitters

In some examples, the transmitter 2101 is an array of multiple NIR LEDs which transmits NIR signals onto the target detection area of the user or subject. In some examples, the transmitter 2101 is configured as an array of more than one near infrared light emitting diodes (NIR LEDs) to emit at least one infrared signal within the near infrared region of the electromagnetic spectrum (from 780 nm to 2500 nm). In some examples, the transmitter 2101 is an array of 3 NIR LEDs which individually emits a first light with a wavelength of 752-1125 nm, a second light with a wavelength of 1080-1620 nm and a third light with a wavelength of 1200-1800 nm, respectively. In this example, the transmitter 2101 is an array of 3 NIR LEDs which individually emits a first light signal with a wavelength of about 940 nm, a second light signal with a wavelength of about 1350 nm and a third light signal with a wavelength of about 1500 nm, respectively. For the case of description, the first light signal, the second light signal and the third light signal (and more light signal) are collectively named as “light signals”, and the first reflected light signal, the second reflected light signal and the third reflected light signal (and more reflected light signals) reflected from the target surface are collectively named as “reflected light signals”. For clarity sake, the reflected light signal reflected from a target surface includes the light components penetrate beneath and reflected from the skin-tissue, blood vessel and blood fluid, etc.

The light emitter and the light receiver of the device 2100 are configured to be positioned over a user's target surface that may have arteries, blood vessels, capillaries, etc. underneath, so that the emitted light signal may direct to the user's arteries, blood vessels, capillaries, etc. and reflected to the light receiver. The emitted lights wave may penetrate through the epidermis, dermis layers and/or the blood vessel walls to the blood fluid of the user. Certain amount of lights will then be absorbed and at least certain remaining amount of the light will be reflected by the blood constituents as well as the body tissues of skin and blood vessel. The reflected light signals will be substantially received by the photodetector 2102.

In this example, NIR LEDs of the transmitter 2101 are switched on and off at different timeslots, back-to-back sequentially to produce light signals separately so as to better capture the absorption and reflection factors of each wavelength by the photodetector 2102. In some examples, the switching of NIR LEDs is driven and controlled by the PCBA 2200.

### Example NIR Photo Detector

Photodetector 2102 is configured to receive the reflected light signals. In this example, photodetector 2102 covers the three detection wavelengths of about 940 nm, about 1350 nm and about 1500 nm. In another example, the photodetector 2102 covers a detection wavelength ranged from 940 nm to 1500 nm. Such arrangement will make the photodetector be able to receive the reflected signals from each of multiple NIR transmitters. In another example, the photodetector 2102 covers a detection wavelength ranged from 940 nm-950 nm. Without being bound by any theory, the attenuation of each reflected light signals will reflect the absorptions due to each constituting blood components. For the attenuation level on each waveform, the level of blood glucose as well as other blood components can be deduced. In some examples, the photodetector 2102 is configured to receive NIR reflected light signals in the range of about 900-1700 nm with a relative spectral sensitivity of at least 40%. In this example, the photodetector 2102 is a monolithic photodiode with an on-chip transimpedance amplifier such as OPT101 (Texas Instruments).

### Example Printed Circuit Board Assembly

Now referring to FIG. 9B, a schematic diagram of the controller, printed circuit board assembly PCBA 2200, is shown. PCBA 2200 is a control system that is configured to operatively connect with the light emitter (transmitter 2101) and the light receiver (photodetector 2102) and aimed to operate the transmitter and the photodetector. The PCBA 2200 is further configured to control the transmitter 2101 to switch on and off to emit the first light signal, the second light signal and the third light signal back-to-back sequentially in a plurality of cycles, such that a plurality of light signal groups, each comprising the first reflected light signal, the second reflected light signal and the third reflected light signal obtained in each cycle are formed at a defined time interval. The PCBA 2200 includes an operating module that controls the operation of the device, and converts the reflected light signals into a digital data. A two-dimensional array consisting of three columns and multiple rows of data vector reflected light signals and In this example, the PCBA 2200 includes a battery management system 2201, a low-dropout regulator (LDO) 2202, a voltage converter 2203, a transmitter connector 2204, a photodetector connector 2205, a wireless module 2206, a user interface indictor 2207, a thermometer 2208, a memory 2209, and a microcontroller unit (MCU) 2210 loaded with an embedded software 2300.

The battery management system 2201 is electrically connected to LDO 2202 and voltage converter 2203 to ensure the power supplies of the PCBA 2200. In some examples, the battery management system 2201 includes a battery (such as a rechargeable battery) and a charger. In some examples, the voltage converter 2203 is a DC/DC converter. In some examples, the voltage of the PCBA 2200 is regulated at 3.3V (3V3).

The voltage converter 2203 is electrically connected to the transmitter connector 2204 and photodetector connector 2205, which are connected to the transmitter 2101 and photodetector 2102, respectively, to supply the regulated voltage to the transmitters 2101 and photodetector 2102. The MCU 2210 is connected to transmitter connector 2204 via general-purpose input/output (GPIO), and the photodetector connector 2205 is connected to MCU 2210 via an analog to digital converter (ADC) interface.

The LDO 2202 is connected to flash memory 2209 and MCU 2210 to ensure the regulated voltage to MCU 2210. MCU 2210 and flash memory 2209 are connected through a serial peripheral interface (SPI) bus.

The MCU 2210 further electrically connects to the wireless module (Bluetooth 5.0) 2206 via BLE 2.4G, the user interface indicator (UI LED) 2207 via GPIO, respectively. The thermometer (NTC) 2208 electrically connects to the MCU 2210 via ADC.

The wireless module 2206 streams the processed signals (digital data vector) to backend console via mobile application (APP) or to backend server via Bluetooth, WiFi, or any other wireless means in the art. In some examples, the wireless module 2206 is Bluetooth 5.0 using a short-range wireless communication protocols, such as those operates in the 2.4 GHz frequency band. In other examples, the wireless module 2206 is replaced by a connection module (e.g., USB connector) that allows direct wired connection instead.

In some examples, the user interface indictor 2207 is connected with the MCU 2210 general-purpose input/output (GPIO) and indicates the battery condition and the detection status of PCBA 2200. The user interface indicator 2207 is controlled by GPIO from MCU 2210.

In some examples, the thermometer 2208 is connected to MCU 2210 via an analog to digital converter (ADC) interface is a negative temperature coefficient (NTC) thermometer which detects the ambience of the system to ensure it is with normal operation range. In some examples, it also captures the subject's body temperature, and the analogue signal is then converted to a further digital data vector for data analysis.

Flash memory 2209 stores the detected and processed photo-detection data (digital data, data vector, digital data vector stream, data vector stream and/or output data, etc.). In some examples, flash memory 2209 is SPI Flash Memory 8M Byte. In other examples, other types and memory sizes of the memory components available in the art may be used instead.

Microcontroller unit (MCU) 2210 is loaded with embedded software 2300 and to execute the control program or embedded software 2300. The embedded software 2300 is a set of software programs designed to carry out operations for the device 2100.

Now referring to FIG. 9C, a flow chart showing the steps of example operations when the embedded software 2300 being executed. The embedded software 2300 contains an operating module controls the operation of the device 2100. In the step 2310, the embedded software 2300 controls the timing of individual NIR LEDs of the transmitter 2101 to emit a signal at a wavelength of 940 nm, 1350 nm or 1500 nm on the target. In some examples, NIR LEDs of the transmitter 2101 are turned ON/OFF at 3 different time-slot back-to-back sequentially to capture the absorption or refection factors of each wavelength.

In the step 2320, the embedded software 2300 controls the receiving timing of photodetector 2102 to receive the reflective signals from the target. In some examples, the reflective signal from the target is 940 nm, 1350 nm or 1500 nm.

In the step 2330, the NIR signals are fed into MCU 2210 for processing.

In the step 2340, the embedded software 2300 processes the light signal groups into digital data vector stream and output to the built in the data processing system in the device having deep meta-learning framework module.

In the step 2350, processes the digital data vector stream by embedded DMLF module 2400.

In the step 2360, displays the results of data analysis, e.g., on the wearable user interface (UI). Additionally or alternatively, the results of data analysis may be reported to other parties such as hospital or care givers by wireless communications.

In other examples, the steps 2350-2360 may be replaced with alternative examples, which will be described in more detail in the below examples 4-5 (for example, data vector streams of 3 wavelength detections will streamed to console via 3206 Bluetooth/WiFi).

### Example Deep Meta-Learning Framework Module

Now referring to FIG. 9D, a flow chart of the DMLF module 2400 of a data processing system is shown. FIG. 9D depicts the software block diagram of the DMLF module 2400 which employs the deep meta-learning framework (also referred as ‘DMLF’) to analyze and predict the blood glucose level in one example of the present invention. Referring to this figure, processed, one or more digital data vector (or near infrared sensor data) is passed to the input data vector module 2410. In some examples, the input data vector further includes or covers other key user varieties or parameters such as genders, ages, races, glycemic conditions, cholesterol levels, and/or across circadian time-stamps. Due to uncontrollable and unavoidable environmental variations, the infrared light vector data may be noisy and contain anomalous data points. Thus, the input data vector is passed to a data preprocessing module 2420 for data preprocessing or cleansing. Afterwards, it is fed to a deep learning ensemble model. In one example, the stacked ensemble model is adopted. The stacked ensemble model comprises at least one basis models 2430. Each of the basis models is selected from a suite of machine learning models and they may be different from each other. The outputs of these basis models are sent to a meta model 2450 via individual model weights denoted as w₁ to wₙ (2440) as shown in FIG. 9D. The meta model 2450 takes in the weighted output values as input and computes an aggregated value which indicates the user's glucose level.

In one example, the input data module 2410 assembles the near infrared sensor data into a two-dimensional array consisting of three columns and multiple rows. Each column corresponds to the reading of the near infrared sensor data at a specific wavelength. The device is designed to take samples of the near infrared sensor data at regular intervals. In an example, the sampling frequency is about 60/minute. Each sample consists of three values corresponding to the readings of the three near infrared sensor data outputs; and they are stored as one row of the aforementioned two-dimensional array. Thus, the array has a large number of rows, and each row has three columns. In some examples, a sliding window (or a sampling time window) can be set to collect a set of (3 column) data vectors, so that there will be 3 mean values and 3 median values, each computed from the respective columns of the data vector collected within the defined period of time. For example, in the case where the sampling “time window” is set to 2 minutes, there will be 120 rows of data, which will be preprocessed and the mean and median for each column will be calculated. In other examples, the time window is set at about 1, 3, 4, 5, 6, 7, 8, 9, 10 or more minutes.

The two-dimensional array (and optionally the respective time-stamps) is passed to the data preprocessing module 2420 to weed out the noisy and anomalous data points. In some examples, this module 2420 performs three subtasks: data cleansing, data processing and/or training data preparation. These subtasks are elaborated below.

The purpose of data cleansing is to remove certain anomalies or outliers from the collected data. These anomalies may occur due to errors in the collected data or because they deviate significantly from the normal data clusters. They can potentially interfere with the proper functioning of deep learning stacked ensemble model during training. In one example, three data cleansing methods are employed. They are: (1) Isolation Forest algorithm (iForest), (2) One-Class Support Vector Machine (SVM) and Local Outlier Factor (LOF) algorithms.

In Isolation Forest (iForest), anomalies are defined as “easily isolated outliers”. It means that they can be considered as points that are far away from densely populated clusters. In the feature space, sparsely populated regions indicate a low probability of events occurring in that area. Therefore, data points falling within these regions are considered anomalous. Isolation Forest is an unsupervised anomaly detection method applicable to continuous data, which means it doesn't require labeled samples for training, but the features need to be continuous. Moreover, Isolation Forest is based on decision trees, which allows it to be easily parallelized and scaled to handle large datasets. It uses an efficient strategy to identify easily isolated points by recursively partitioning the dataset until all samples are isolated. With this random partitioning strategy, anomalous points typically have shorter paths and can then be filtered out.

The One-Class SVM algorithm shares similar principles and mathematical models with the SVM algorithm. It treats the entire data set as a single class and seeks a hyperplane that separates normal data from anomalous data. This hyperplane is defined as the one that maximizes the margin between the boundary and the observations in the class. During the testing phase, new data points are evaluated to determine whether they are inside or outside the boundary. Points that fall outside the boundary are classified as outliers.

Local Outlier Factor (LOF) is a probabilistic model commonly used for clustering or density estimation of data. In the context of outlier detection, LOF can identify outlier points by estimating the probability density distribution of the data. Outlier points typically have lower probability densities, so their status as outliers can be determined by calculating the probability of data points under each Gaussian distribution.

The performances of the three data cleansing methods were evaluated and shown in Table-1 below. These experiments were carried out under the condition that except for the internal parameters of these three methods, all other parameters in the data preprocessing module as well as the stacked ensemble models including the basis models and the meta model were fixed. The same dataset were used to evaluate the three cleansing methods. 1240 volunteers were recruited to evaluate the accuracy of the example non-invasive continuous glucose monitor system 2000 and device 2100 (abbreviated as ‘niCGM’ hereinafter) and compare it with the performance of a standard hospital clinical device BS-350E (Mindray) which obtains the blood glucose level by drawing blood (or pricks a finger using a needle to collect blood) from a volunteer and analyzing the blood sample in a laboratory to obtain information (abbreviated as ‘control’ hereinafter). The test data were measured from volunteers #1-1240. 70% of the data were used for training of the DMLF module and 30% of the data were used for system performance evaluation. Hence the performance measure is the accuracy between the predicted values against the verified data. The accuracy of the example device is calculated by the mean absolute relative difference (MARD). For example, if the compared value is x, the reference value is y, then the absolute relative difference is |x−y|/y*100%, and MARD is the average of all the absolute relative differences.

In these experiments, the following parameters are used for each data cleansing method. For Isolated Forest: the number of estimator is 100; sample size is 256; maximum number of features is 1 and outliner ratio is 0.1. For One-Class SVM, the outliner ratio is set to 0.1; and for LOF, it is set to detect 20 local outlier factor points with an outlier ratio of 0.2 using the Euclidean distance metric. With this set of parameters, each data cleansing method is evaluated alone and in combination with others. As shown in Table-1, the best result was obtained by combining the Isolation Forest and One-Class SVM together. It yields an accuracy of 0.882.

TABLE 1
Data Cleansing Experimental results

| Data cleaning methods | | | |
|---|---|---|---|
| Isolation forest | One Class SVM | LOF | ACCUARCY |
| Yes | | | 0.795 |
| | Yes | | 0.801 |
| | | Yes | 0.782 |
| Yes | Yes | | 0.882 |
| Yes | | Yes | 0.876 |
| | Yes | Yes | 0.869 |
| Yes | Yes | Yes | 0.863 |

Based on the experimental results, both the iForest and the One-Class SVM are used for the data cleansing subtask. In one implementation, both modules process the data from data vector module 2410 sequentially. If a data point is identified as outliner or anomalous by the first method it will be discarded from the dataset for subsequent processing. In other examples, the modules are performed concurrently. If any data point is identified as outliner or anomalous by either one or both methods it will be discarded from the dataset for subsequent processing.

After data cleansing, the purpose of data processing is to consolidate the data within a table. Common practices involve using the mean or median of each dimension (i.e. each column of the dataset table). In one example, the mean and median of each column of the data table is computed. In a further example, the approach of representing the data as the average of the median and mean values is adopted. Table-2 shows the results of three experiments. i.e. mean alone, median alone and the average of mean and median. Again, the experimental conditions are the same as before and will not be repeated here.

TABLE 2
Experimental results of data processing method

| Data Processing method | Accuracy |
|---|---|
| Mean | 87.3 |
| Median | 87.5 |
| (Mean + Median )/2 | 88.2 |

Table-2 shows that by taking the average of the mean and median, the accuracy improves. Hence this approach is adopted in one implementation of this invention.

The third subtask of the data preprocessing module 2420 is training data preparation. In one example, the bootstrap sampling approach is adopted. In essence, after data cleansing and taking the average of the mean and median values, a new data subset is generated for each of the basis models 2430. These data subsets are generated randomly by a sampling with replacement methodology. Each subset has the same size as the original dataset, but some instances may be duplicated while others may be left out. As such, each data subset is unique and different from the others.

The data subsets are then passed to a stacked ensemble model which consists of at least two layers. In an example depicted in FIG. 9D, the first layer comprises a plurality of basis models 2430 and the second layer is a meta model 2450. Each basis model in the first layer is connected to the meta model via a model weight 2440. The stacked ensemble model is a powerful and flexible machine learning method that can combine the prediction results of individual basis model to achieve a better and more robust prediction.

In an examples for blood glucose level prediction, each basis model acts as a regressor that takes a data subset assigned to this basis model as input and computes a value representing the blood glucose level. This value is a continuous variable. The basis model can be selected from a variety of deep learning regressors. In one example, the candidate for the basis model includes, but is not limited to, the Recurrent Neural Network (RNN) regressor, Long Short-Term Memory (LSTM) regressor, Convolution Neural Network (CNN) regressor, Support Vector Regression (SVR), Multi-Layer Perceptron (MLP) regressor, k-Nearest Neighbors (KNN) model, ElasticNetCV regressor, Catboost regressor, XGBoost (eXtreme Gradient Boosting) regressor, Gradient Boosting Regressor, LGBM regressor, Bagging regressor, decision tree . . . , etc.

In one example, the same deep learning regressor may be deployed to one or more basis models 2430. As the data preprocessing module 2420 prepares different data subsets for each of these basis models, even when different basis models choose the same deep learning regressor, these basis models are trained to capture different aspects of the overall data set. Besides, the stacked ensemble adopts a flexible framework whereby different basis models can select different deep learning regressors. In some cases, a basis model may by itself be a stacked ensemble model. Hence the ensemble of basis models is capable of covering all aspects of the infrared sensor data. By combining multiple predictions together at the meta model 2450, the overall prediction can be substantially improved.

Many aforementioned the deep learning techniques require training before they are put to use. Training is a process that by repeatedly presenting a set of training data to the basis model, a learning algorithm specifically designed for that deep learning regressor is able to adjust the internal parameters to improve its prediction performance. In one example, the data subset is partitioned to a training set and a test set. In a further example, 70% of the data subset is used for training and the remaining 30% for testing. After the basis model is properly trained, the internal parameters are frozen, and the basis models are readily available to perform regression prediction.

The output of individual basis model 2430 is sent to input of the meta model 2450 via the respective model weight 2440. The model weights 2440 may be the same in one example. They may be randomly assigned in another examples. In yet another example, they may be trained. In one implementation, after the basis models are trained and their respective internal parameters frozen, the same set of training data is fed to the basis models. The output of each basis model acts as the input of these model weights. In a further implementation, the PyTorch machine learning library is used to define a neural network model and its trainable parameters. The nn.Parameter subclass in this library is used train the model weights 2440. This is done by initializing all model weights to ones (1) with torch.ones first. Then they are wrapped with nn.Parameter. This allows the model weights to be trained synchronously with the model's gradient descent during the training process.

In a similar fashion, the meta model 2450 may be trained after the basis models 2430 and the model weights 2440 are trained and frozen. The same training data set, with same partition of 70% training and 30% testing, is used to train the internal parameters of the meta model. In one example, the candidate for the meta model includes, but is not limited to, the Multi-Layer Perceptron (MLP), the Recurrent Neural Network (RNN), the Convolution Neural Network (CNN), etc.

A suite of experiments was conducted to evaluate the performance of the MDLP module. The experimental setup is the same as before and will not be repeated here. In these experiments, we focus on the performance of the stacked ensemble model, i.e. the development of basis models and meta models, while keeping the parameters in the data preprocessing module unchanged. Table 3 below shows the results.

TABLE 3
Experimental results of the MDLP module which
implements the deep Meta Learning Framework

| Basis models | Meta model | ACCURACY |
|---|---|---|
| SVR + MLP + Catboost | Single Layer MLP | 0.888 |
| SVR + MLP + Catboost | Two Layer MLP | 0.876 |
| SVR + MLP + XGboost | Single Layer MLP | 0.882 |
| SVR + Catboost | Two Layer MLP | 0.869 |
| SVR + KNN + Catboost | Single Layer MLP | 0.857 |
| SVR + KNN + Catboost + | Single Layer MLP | 0.851 |
| MLP | | |
| MLP + Catboost + KNN | Single Layer MLP | 0.819 |
| KNN + Catboost + | Single Layer MLP | 0.845 |
| ElasticNetCV | | |
| SVR + MLP + XGboost | Single Layer MLP | 0.851 |

As shown in Table 3, the first layer of the stacked ensemble model comprises three or four basis models. They are selected from SVR, MLP, Catboost, XGBoost, ElasticnetCV and KNN. The parameters for each of these regression models are as follows:

For SVR, Gaussian kernel function with a control coefficient of 0.4 for the number of support vectors is used. The tolerance stopping criterion is set to 0. 0001 while the error term penalty parameter is 0.2. The specified kernel cache size is 1999 and the maximum iteration count is set at 38000.

For Catboost, the iteration count is set at 600 and the learning rate at 0.2. The maximum depth of each tree is 8 and a Root Mean Square Error (RMSE) loss function is chosen. The bagging aggressive coefficient is 0.2 and the overfitting detection type is set to ‘Iter’ while the overfitting detection waiting time is set at 100.

For XGBoost, the learning rate is set at 0.2 while the maximum depth of each tree is set at 6. The number of learners is 150. The ‘gbtree’ tree model is used as the base estimator and the penalty coefficient is set at 1.

For ElasticnetCV, the L1 norm is set at 0.5 and path length set at 1×10⁻³. For each L1 regularization path, 100 alpha values are tried. The maximum interaction count is set at 1000 and the tolerance stopping criterion set at 0.00001.

For KNN, the number of categories is set at 17. In other words, the output value is calculated as the average of the nearest 17 values.

For the basis model MLP, the complete architecture is shown in FIG. 9E. In one example, one or more of the basis model is a Multi-Layer Perceptron (MLP) regressor. FIG. 9E depicts a basis model MLP neural network architecture 2600. It comprises an input layer 2610, zero or more hidden layers and an output layer 2680. In FIG. 9E, a network with two hidden layers 2630 and 2650 are shown. Each of the input layer 2610, hidden layers 2630 and 2650 has a plurality of nodes, while the output layer 2680 has one node as this MLP model is configured as a regressor. In this example, there are three nodes in the input layer 2610, and there are six nodes in each hidden layers 2630 and 2650, respectively. In this basis model MLP, the outputs of the first hidden layer 2630 and the outputs of the second hidden layer 2650 are summed together at the summation point 2660. The nodes between the respective layers are fully connected, meaning that each node in the first hidden layer 2630 is connected to all the nodes in the input layer 2610. Similarly, each node in the second hidden layer 2650 is connected to all the nodes in the first hidden layer 2630; and the output node 2680 is fully connected to outputs of the summation 2660. Labels 2620, 2640 and 2670 represent the respective fully connected network. Each connection is associated with a weight parameter which is trainable. Except the input nodes, all hidden nodes and the output node may also have a bias parameter which is also trainable. In operation, a data vector is first fed to the input layer, and then propagated through the hidden layers by computing a weighted sum of the inputs with the connection weights and applying an activation function to produce the output of each node, until the final output is produced by the output layer. To train the MLP regressor, the output node value is then compared with a desired value, and the error is then back-propagated towards the input layer. Then the connection weights and the bias parameters are adjusted to minimize the error. By repeating this process multiple times, the MLP regressor is trained to yield a regression value with high accuracy.

As for the meta model, either a single layer MLP as shown in FIG. 9F or a two-layer MLP as shown in FIG. 9G is chosen. In FIG. 9F, the input layer 2710 comprises three input nodes. There is no hidden layer. The output node 2730 is connected to each of the input nodes via connections 2720. In FIG. 9G, both the input layer 2740 and the hidden layer 2760 have three nodes. All these nodes are fully connected to each other via connections 2750. The single output node 2780 is also fully connected to the hidden nodes 2760 via connections 2770. Furthermore, each hidden nodes and the output node are also embedded with an activation function. Various activation functions may be used, including but not limited to, the Rectified Linear Unit (ReLU), Leaky ReLU, Tanh (hyperbolic tangent), and the Sigmoid function, . . . , etc.

From the experimental results shown in Table 3, the stacked ensemble model that yields the highest accuracy comprises a first layer of three basis models chosen from SVR, MLP and Catboost; and a single layer MLP regressor using ReLU activation function as a meta model. This combination achieves the highest accuracy of 0.888 from the experimental data set.

It would be appreciated that the third subtask in the data preprocessing module-training data preparation is only needed to generate data subsets for the purpose of training the basis models, the model weights and the meta model. Once these models are training, and the DMLF module 2400 is put to actual use, there is no need to perform this subtask so it can be skipped.

### Example 3

### Example Blood Glucose Monitoring System and Device

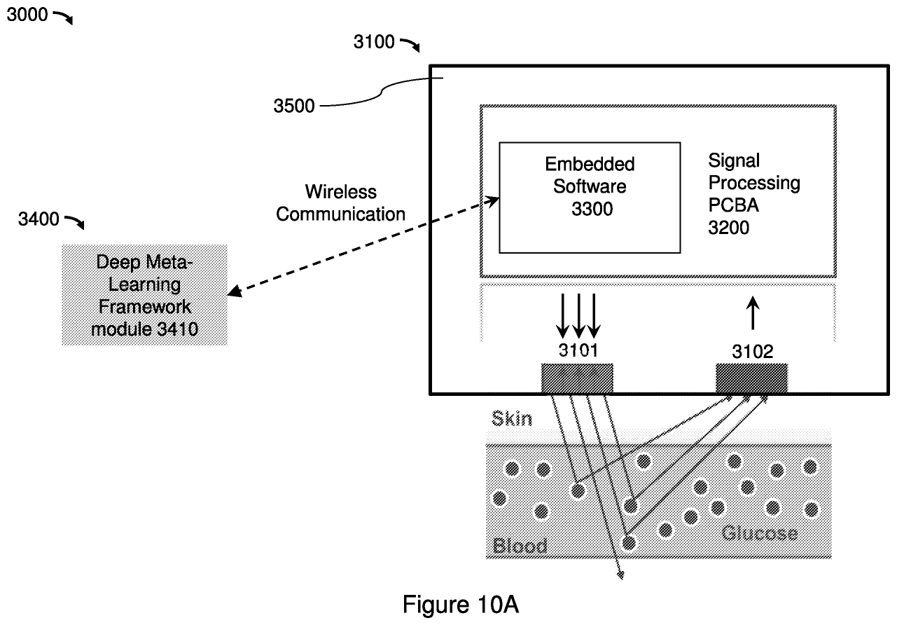

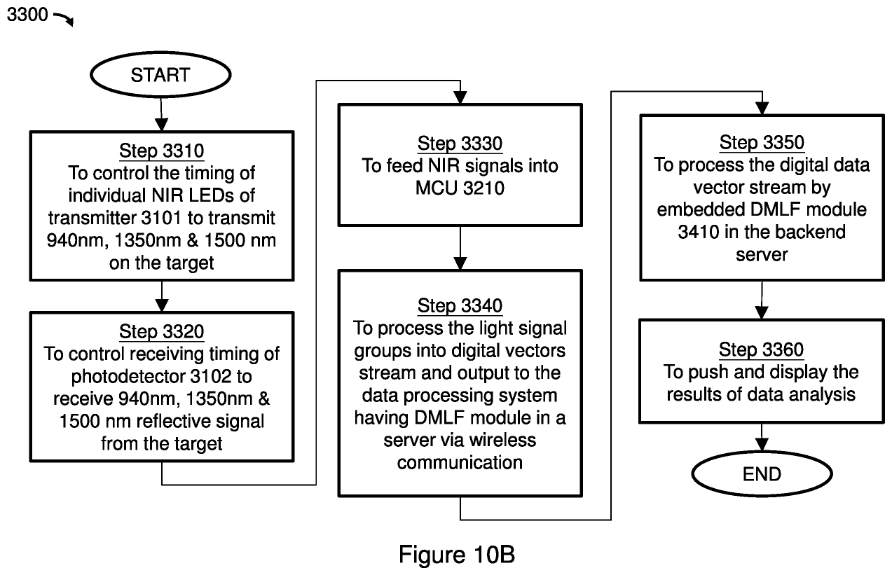

Now referring to FIG. 10A, a schematic diagram of another example blood glucose monitoring system 3000 comprising a device 3100 of another example non-invasive continuous glucose monitor (niCGM) and a server 3400 is shown. Similar to Example 2, the device 3100 includes a housing body 3500, a transmitter 3101, a photodetector 3102, and a signal processing PCBA 3200 disposed within the housing body 3500. In this example, the PCBA 3200 includes embedded software 3300, which is indirectly connects to a remote server 3400 containing DMLF module 3410 in via wireless communication.

Now referring to FIG. 10B, a flow chart showing the steps of example operations when the embedded software 3300 being executed. The embedded software 3300 contains an operating module controls the operation of the device 3100. In the step 3310, the embedded software 3300 controls the timing of individual NIR LEDs of the transmitter 3101 to emit a signal at a wavelength of 940 nm, 1350 nm and 1500 nm on the target. In some examples, NIR LEDs of the transmitter 3101 are turned ON/OFF at 3 different time-slot back-to-back sequentially to capture the absorption or refection factors of each wavelength.

In the step 3320, the embedded software 3300 controls the receiving timing of photodetector 3102 to receive the reflective signals from the target. In some examples, the reflective signal from the target is 940 nm, 1350 nm and 1500 nm.

In the step 3330, the NIR signals are fed into MCU 3210 for processing.

In the step 3340, the embedded software 3300 processes the light signal groups into digital data vector stream and output to the data processing system having deep meta-learning framework module in a server via wireless communication (e.g., by WiFi).

In the step 3350, processes the digital data vector stream by embedded DMLF module 3410 in the backend server.

In the step 3360, pushes and displays the results of data analysis from server to a display element, e.g., on the wearable user interface (UI). Additionally or alternatively, the results of data analysis may be reported to other parties such as hospital or care givers by wireless communications.

### Example 4

### Example Blood Glucose Monitoring System and Device

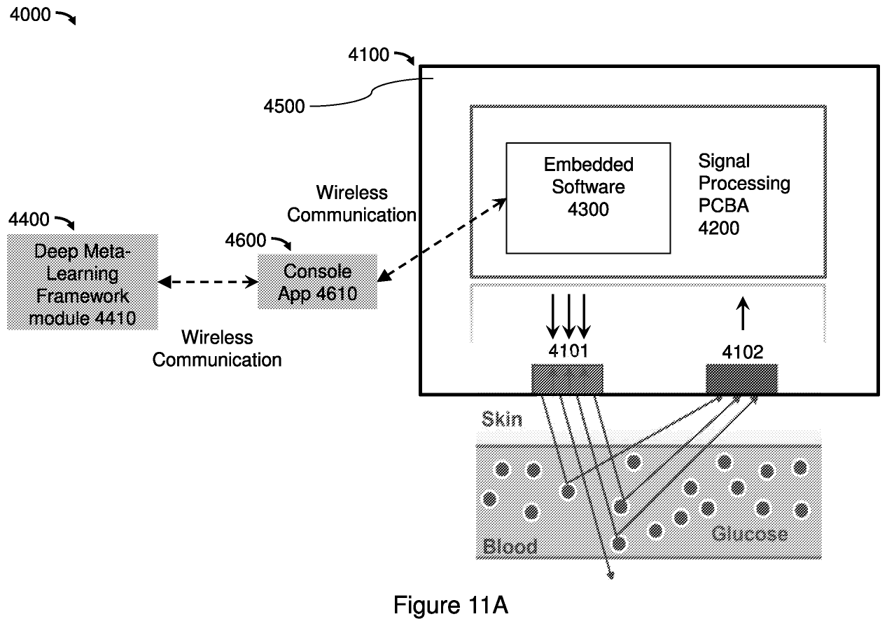

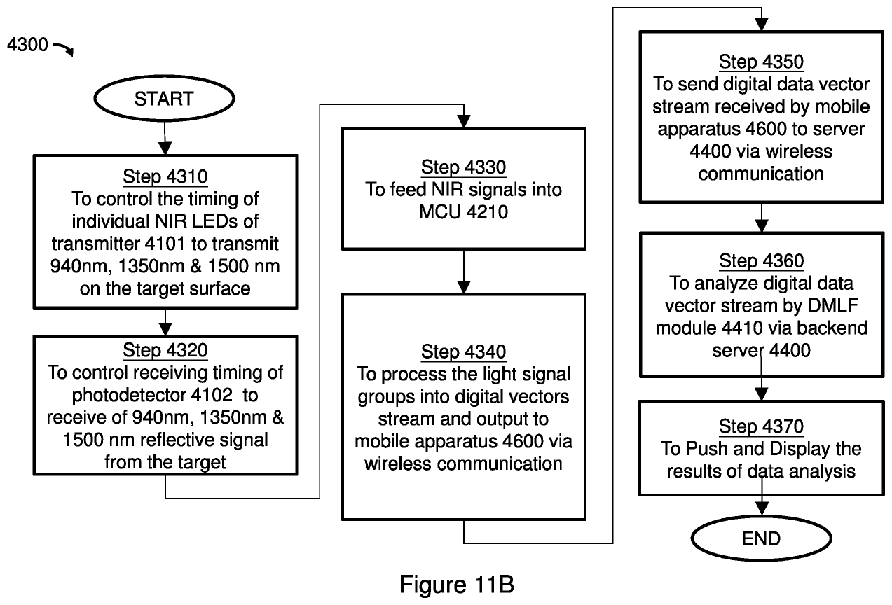

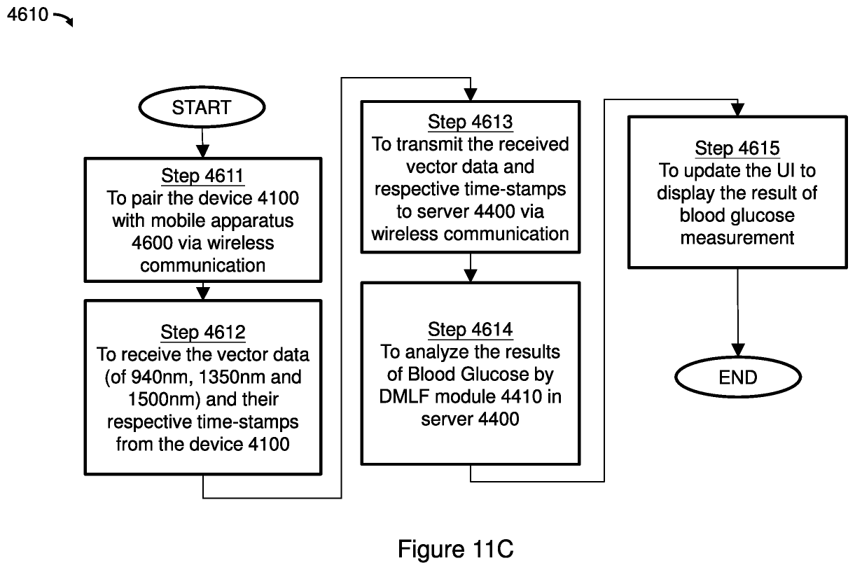

Now referring to FIG. 11A, a schematic diagram of another example blood glucose monitoring system 4000 comprising a device 4100 of another example non-invasive continuous glucose monitor (niCGM), a mobile apparatus 4600 containing console APP 4610 and a remote server 4400 containing DMLF module 4410 is shown. Similar to the preceding examples, the device 4000 includes a housing body 4500, a transmitter 4101, a photodetector 4102, and a signal processing PCBA 4200 disposed within the housing body 4500, similar to the device 2100 as described in Example 2. In this example, the PCBA includes embedded software 4300, which indirectly connects to a console APP 6410 in the mobile apparatus 4600 via wireless communication (e.g., Bluetooth). The mobile apparatus 4610 further connects to the DMLF 4410 of a server 4400 via wireless communication (e.g., WiFi). The mobile apparatus 4610 can be a mobile phone, laptop, or replaced by a desktop computer.

Now referring to FIG. 11B, a flow chart showing the steps of example operations when the embedded software 4300 being executed. The embedded software 4300 contains an operating module controls the operation of the device 4100. In the step 4310, the embedded software 4300 controls the timing of individual NIR LEDs of the transmitter 4101 to emit light signals at a wavelength of 940 nm, 1350 nm and 1500 nm on the target surface. In some examples, NIR LEDs of the transmitter 3101 are turned ON/OFF at different time-slot back-to-back sequentially to capture the absorption or refection factors of each wavelength.

In the step 4320, the embedded software 4300 controls the receiving timing of photodetector 4102 to receive the reflective signals from the target surface. In some examples, the reflected light signals from the target surface is about 940 nm, about 1350 nm and about 1500 nm.

In the step 4330, the NIR light signals are fed into MCU 4210 for processing into the digital data vector stream in the PCBA 4200 and output the same to the console APP 4610 in mobile apparatus 4600 via wireless communication (e.g., bluebooth).

In the step 4340, the embedded software 4300 in the PCBA 4200 processes the light signal groups into digital data vector stream and output to mobile apparatus 4600 (e.g., console APP 4610), via wireless communication (e.g., by WiFi).

In the step 4350, the digital data vector stream received by the mobile apparatus 4600 (e.g., console APP 4610) are sent to server 4400 via wireless communication.

In the step 4360, the digital data vector stream are analyzed by embedded DMLF module 4410 in the backend server the backend server 4400.

In the step 4370, the results of data analysis from server are pushed to and displayed by a display element, e.g., on the wearable user interface (UI) and/or the mobile apparatus. Additionally or alternatively, the results of data analysis may be reported to other parties such as hospital or care givers by wireless communications.

Now referring to FIG. 11C, a flow chart showing the steps of example overall operations of the console APP 4610 of the blood glucose monitoring system to provide updated blood glucose level continuously or regularly.

In the step 4611, the device 4100 is paired with the console APP 4610 via wireless communication (e.g., Bluetooth).

In step 4612, the vector data and their respective time-stamps from the device 4100 were received by the paired mobile apparatus 4600. In one implementation, the data vector are processed by the device 4100 before sending to the console APP 4610.

In step 4613, the received vector data and their respective time-stamps were transmitted to the server 4400 via wireless communication (e.g., WiFi).

In step 4614, the transmitted vector data (including the time-stamp information) were analyzed by DMLF module 4410 in the server 4400 to produce output data results of blood glucose level.

In step 4615, based on the output data results, the display element (such as a UI in the device or console APP) was updated to display the output data results of blood glucose level. In one implementation, the UI in the device 4100 were used to display the latest output data results of blood glucose level.

### Example 5

### Device and System Operating Procedures

In this example, the general procedures how to operate the blood glucose monitoring devices and systems thereof as described in previous Examples 1-4 are described below.

To measure the blood glucose level of a user, the blood glucose monitoring device is placed on a target surface (e.g., fore-arm) of the user, with the NIR transmitter and the photodetector facing the target surface. For better results, ensure the NIR transmitter and the photodetector are in good contact with (e.g., stick firmly onto) the target surface.

The device is turned on, such that the NIR transmitter emits the light signals onto the target surface, and the reflected light signals are received by the photodetector. In some examples, the multiple light signals (or signal streams) are emitted back-to-back sequentially, such that multiple reflected light signals are received. In some examples, the device is turned on to capture data for about 2 minutes for data collection. In some examples, the device sends an alert alarm upon completion of data (reflected light signal) collection. In some examples, certain data can be manually or automatically selected for subsequent analysis. For example, certain data can be selected after the readings are stabilized.

The reflected light signals are processed in the operating module in the controller into digital data vector. In some examples, reflected light signals are received regularly or continuously, and they are processed into data vector streams. Then, the data vector (or data vector streams) are either sent directly to the data processing system built-in in the controller, or sent indirectly to a computer or a backend server having the data processing system for data processing. The data processing system includes a trained machine learning module (e.g., the DMLF module) to analyze the data vector into output data as blood glucose level of the user. In some examples, the data vector are sent to backend server indirectly via a mobile apparatus such as by wireless communication.

After data analysis, the output data may be directly or indirectly sent to mobile apparatus, the device or other display means to report the blood glucose level of the user.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

Devices/methods/methods discussed within different figures can be added to or exchanged with those in other figures. Further, specific numerical data values (such as specific quantities, numbers, categories, etc.) or other specific information should be interpreted as illustrative for discussing example embodiments. Such specific information is not provided to limit example embodiment.

For example, in certain embodiments, light emitter is configured to emit three light signals (with wavelengths of about 940 nm, about 1350 nm and about 1500 nm, but different numbers (e.g., one, two, three, four, five, six, seven, eight, nine, ten or more) of light signals, with different wavelengths, different emitting order and/or different time intervals/frequencies may be used according to the practical need. For example, the light emitter can be configured to additionally emit one or more (say, fourth, fifth, sixth, seventh, eighth, ninth, tenth or more) light signals, each additional light signal has a wavelength selected from the range of about 400 nm-2000 nm, respectively. For example, the wavelength is 400 nm, 450 nm, 500 nm, 550 nm, 600 nm, 650 nm, 700 nm, 750 nm, 800 nm, 850 nm, 900 nm, 950 nm, 1000 nm, 1050 nm, 1100 nm, 1150 nm, 1200 nm, 1250 nm, 1300 nm, 1350 nm, 1400 nm, 1450 nm, 1500 nm, 1550 nm, 1600 nm, 1650 nm, 1700 nm, 1750 nm, 1800 nm, 1850 nm, 1900 nm, 1950 nm, or 2000 nm.

For example, in certain embodiments, WiFi and bluetooth are used as wireless communication between different components, but other wireless communication means such as infrared, zig-bee, near field communication etc. may also be used instead.

For example, in certain embodiments, the battery management system includes a battery (such as a rechargeable battery) and a charger, but it can include a connector (such as USB connector) instead to connect with a external power source externally, for example, a DC power source.

### NUMBERED EMBODIMENTS

### Set 1

Embodiment 1. A glucose monitoring system comprising: a wearable device capable of measuring blood glucose levels in a user, the device comprising: a housing body, an optical sensor comprising a circuit including a near infrared light emitting diode and a receiver chip and configured to produce a voltage signal, and a processing unit configured to convert the analog voltage signal to a digital voltage signal; and computer software comprising algorithms producing trained neural network models capable of predicting the user's blood glucose level in real time based on the voltage signals received from the processing unit wherein the system is configured to non-invasively measure the user's blood glucose levels in real time.

Embodiment 2. The glucose monitoring system of any one of the preceding embodiments, wherein the optical sensor is connected to the processing unit by the circuit.

Embodiment 3. The glucose monitoring system of any one of the preceding embodiments, wherein the trained neural network models are part of the processing unit.

Embodiment 4. The glucose monitoring system of any one of the preceding embodiments, wherein the trained neural network models are stored in a device or network separate from the device.

### Set 2

Embodiment 1: A glucose monitoring system comprising: a wearable device capable of measuring blood glucose levels in a user, the device comprising: a housing body, an optical sensor comprising a circuit including a near infrared light emitting diode and a receiver chip and configured to produce a voltage signal, and a processing unit configured to convert the analog voltage signal to a digital voltage signal; and computer software comprising algorithms producing trained neural network models capable of predicting the user's blood glucose level in real time based on the voltage signals received from the processing unit, wherein the system is configured to non-invasively measure the user's blood glucose levels in real time, wherein the trained neural network models comprises a trained non-liner model and a linear model to execute the following steps:
producing a class prediction probability value and a numerical value, by subjecting the voltage signals to the trained non-linear model and the linear model, respectively;
classifying the class prediction probability value and the numerical value as being low, normal or high;
comparing the classification results to determine if the values are consistent; and
if the values are consistent, determining the output blood glucose state and the blood glucose value.

Embodiment 2. The glucose monitoring system of any one of the preceding embodiments, wherein the optical sensor is connected to the processing unit by the circuit.

Embodiment 3. The glucose monitoring system of any one of the preceding embodiments, wherein the trained neural network models are part of the processing unit.

Embodiment 4. The glucose monitoring system of any one of the preceding embodiments, wherein the trained neural network models are stored in a device or network separate from the device.

### Set 3

Embodiment 1. A device for blood glucose monitoring of a user, comprising: (a) a light emitter, configured to emit a light signal directed to a target surface of the user, so as to generate a reflected light signal that is reflected from the target surface; (b) a light receiver, configured to receive the reflected light signals; (c) a controller, configured to operatively connect with the light emitter and the light receiver; and (d) an enclosure, configured to receive the light emitter, the light receiver and the controller, wherein the light signal comprises a first light signal having a first wavelength of about 940 nm, a second light signal having a second wavelength of about 1350 nm, and/or a third light signal having a third wavelength of about 1500 nm, wherein the controller comprises an operating module that controls the operation of the device, and converts the reflected light signal into a digital data, and wherein the controller further comprises or operatively connects with a data processing system that processes the digital data, wherein the data processing system comprises a machine learning module that analyzes the data signal to generate an output data that is a blood glucose level of the user.

Embodiment 2. The device of any one of the preceding embodiments, wherein the light emitter comprises three near infrared LEDs having the first wavelength, the second wavelength and the third wavelength, respectively.

Embodiment 3. The device of any one of the preceding embodiments, wherein the device further comprises a NTC thermometer to obtain ambient temperature and/or the user's body temperature.

Embodiment 4. The device of any one of the preceding embodiments, wherein the controller is further configured to control the light emitter to switch on and off to emit the first light signal, the second light signal and/or the third light signal back-to-back sequentially in a plurality of cycles, such that a plurality of light signal groups, each comprising the first reflected light signal, the second reflected light signal and/or the third reflected light signal obtained in each cycle are formed at a defined time interval.

Embodiment 5. The device of any one of the preceding embodiments, wherein the time interval is about 60 times per minute.

Embodiment 6. The device of any one of the preceding embodiments, wherein the controller comprises a processor unit coupled with a memory that stores an executable, software program, the software program comprises an operating module that controls the operation of the device, wherein the operation system executes the following steps: a) controlling timing of the light emitter to switch on and off to emit the light signal; b) controlling timing of light receiver to receive the reflected light signal to obtain a plurality of reflected light groups; c) processing individual reflected light signal group into a digital, data vector; and d) transmitting the data vector to a data processing system to analyze the data vector.

Embodiment 7. The device of any one of the preceding embodiments, wherein the machine learning module comprises a deep meta learning framework (DMLF) module that processes the data vector obtained from the device to generate an output data.

Embodiment 8. The device of any one of the preceding embodiments, wherein the data processing system executes the following steps: a) obtaining the data vector from the device; b) pre-processing the data vectors to produce processed data vector; and c) analyzing the processed data vector by a trained DMLF module to produce an output data as a blood glucose level of the user.

Embodiment 9. The device of any one of the preceding embodiments, wherein the step b) comprises the step of: b1) cleaning the data vectors to remove any outlier.

Embodiment 10. The device of any one of the preceding embodiments, wherein the step b1) is performed by using isolation forest (iForest), one class SVM and LOF, and combination thereof.

Embodiment 11. The device of any one of the preceding embodiments, wherein the step b1) is performed by a combination of isolation forest and one class SVM.

Embodiment 12. The device of any one of the preceding embodiments, wherein the step b) comprises the step of: b2) consolidating a data vector into a representing data by the following equation: representing data=(y+z)/2, wherein y is the mean value of the data vector and z is the median value of the data vector.

Embodiment 13. The device of any one of the preceding embodiments, wherein the DMLF module employs a deep meta-learning framework to analyze the data vector, comprising a first hierarchical layer and a second hierarchical layer, the first hierarchical layer comprising a plurality of basis models, and the second hierarchical layer comprising a meta model, wherein each basis models is configured to receive a processed data vector and produces an intermediate data point, and the meta model is configured to receive a weighted value of the intermediate data point to produce the output data.

Embodiment 14. The device of any one of the preceding embodiments, wherein the basis model is selected from a group consisting of RNN, LSTM, CNN, Support Vector Regression (SVR), MLP, KNN, ElasticNetCV, Catboost, XGBoost, Gradient Boosting Regressor, LGBM regressor, Bagging regressor, decision tree, XGBoost, and combination thereof.

Embodiment 15. The device of any one of the preceding embodiments, wherein the meta model is selected from MLP, RNN, CNN, and any combination thereof.

Embodiment 16. The device of any one of the preceding embodiments, wherein weighted value of the intermediate data point is computed by multiplying the intermediate data point by a model weight wherein each model weight is configured to be the same value, a random value or an optimized value obtained by a learning algorithm.

Embodiment 17. The device of any one of the preceding embodiments, wherein first hierarchical layer comprises basis model of SVR, MLP and Catboost, and a meta model configured as a single layer MLP using ReLU as its activation function.

Embodiment 18. The device of any one of the preceding embodiments, wherein the DMLF module is pre-trained by a set of training data using boostrap sampling with a sampling with replacement methodology.

Embodiment 19. A device for blood glucose monitoring of a user, comprising: a) a light emitter, having a plurality of near infrared LEDs, configured to emit a light signal directed to a target surface of the user, so as to generate a reflected light signal that is reflected from the target surface, respectively, wherein the light signal comprises a first light signal having a wavelength of about 940 nm, a second light signal having a wavelength of about 1350 nm and a third light signal having a wavelength of about 1500 nm; b) a light receiver, having a photo-detector, configured to receive the reflected light signals; and c) a controller, configured to operatively connect with the light emitter and the light receiver; and d) an enclosure, configured to receive the light emitter, the light receiver and the controller, wherein the controller is configured to control the light emitter to switch on and off to emit the first light signal, the second light signal and the third light signal back-to-back sequentially, such that a plurality of reflected light groups, each comprising the first reflected light, the second reflected light and the third reflected light obtained in each cycle are formed at a defined time interval, wherein the controller comprises a processor unit coupled with a memory that stores an executable, software program, the software program comprises an operating module that controls the operation of the device, and converts individual reflected light groups into a digital data vector, and wherein the controller further comprises or operatively connects with a data processing system that processes the data vector, wherein the data processing system comprises a machine learning module that analyzes the data vector to generate an output data that is a blood glucose level of the user.

Embodiment 20. The device of any one of the preceding embodiments, wherein the software program comprises an operating module that controls the operation of the device, wherein the operating module executes the following steps: a) controlling timing of the light emitter to switch on and off to emit the first light, the second light and the third light; b) controlling timing of light receiver to receive the first reflected light, the second reflected light and the third reflected light that are reflected from the target surface to generate a plurality of reflected light groups; c) processing individual reflected light group into a digital, data vector; and d) transmitting the data vector to a data processing system comprising a neural network to process the data vectors

Embodiment 21. The device of any one of the preceding embodiments, wherein the machine learning module comprises a deep meta learning framework module, comprising a first hierarchical layer and a second hierarchical layer, the first hierarchical layer comprises a plurality of basis modules, the second hierarchical layer comprises a meta learning module, wherein each basis module is configured to receive a processed data vector and produces an intermediate data point, the meta learning module is configured to receive a weighted value of the intermediate data point to produce the output data, wherein first hierarchical layer comprises basis modules of SVR, MLP and Catboost, and second hierarchical layer comprises a meta learning module of ReLU.

Embodiment 22. A system for blood glucose monitoring of a user, comprising: (a) a device as claimed in any one of the preceding embodiments; and (b) a server in electrical communication with the device.

Embodiment 23. The system of any one of the preceding embodiments, wherein the server comprises a server processor unit coupled with a server memory that stores an executable server software program, the server software program comprises a data processing system that processes a data vector obtained from the device to calculate the blood glucose level of the user, wherein the data processing system comprises a neural network.

Embodiment 24. The system of any one of the preceding embodiments, wherein the data processing system executes the following steps: a) obtaining the data vectors obtained from the device; b) pre-processing the data vectors; and c) analyzing the data vector by trained neural network model to produce an output data, such that a blood glucose level of the user is obtained

Embodiment 25. The system of any one of the preceding embodiments, wherein the step b) comprises the step of: b1) cleaning the data vectors to remove any outlier.

Embodiment 26. The system of any one of the preceding embodiments, wherein the step b1) is performed by using isolation forest (iForest), one class SVM and LOF, and combination thereof.

Embodiment 27. The system of any one of the preceding embodiments, wherein the step b1) is performed by a combination of isolation forest and one class SVM.

Embodiment 28. The system of any one of the preceding embodiments, wherein the step b) comprises the step of: b2) consolidating a vector data into a representing data by the following equation: representing data=(a+b)/2, wherein x is the average of the vector data and y is the median of the vector data.

Embodiment 29. The system of any one of the preceding embodiments or claim 23, wherein the neural network is a deep meta learning framework, comprising a first hierarchical layer and a second hierarchical layer, the first hierarchical layer comprises a plurality of basis modules, the second hierarchical layer comprises a meta learning module, each basis module is configured to receive a data vector and produces an intermediate data vector, the meta learning module is configured to receive the intermediate data to produce the output data.

Embodiment 30. The system of any one of the preceding embodiments, wherein individual basis modules are machine learning modules or deep learning models.

Embodiment 31. The system of any one of the preceding embodiments, wherein the basis module is selected from a group consisting of RNN, LSTM, CNN, Support Vector Regression (SVR), MLP, KNN, ElasticNetCV, Catboost, XGBoost, Gradient Boosting Regressor, LGBM regressor, Bagging regressor, decision tree, XGBoost, Stacking, and combination thereof; and the meta learning module is selected from ReLU, Leaky ReLU, Tanh, Sigmoid, and combination thereof.

Embodiment 32. The system of any one of the preceding embodiments, wherein each basis module comprises a model weight, each model weight is configured to be the same value, a random value or optimized by using the nn.Parameter function in Pytorch.

Embodiment 33. The system of any one of the preceding embodiments, wherein first hierarchical layer comprises basis modules of SVR, MLP and Catboost, and second hierarchical layer comprises a meta learning module of ReLU.

Embodiment 34. The system of any one of the preceding embodiments, wherein the neural network is pre-trained by training data using boostrap sampling.

Embodiment 35. The system of any one of the preceding embodiments, further comprising a mobile apparatus that is in electrical communication between the device and the server, configured to receive a data vector obtained from the device, to transmit the data vector to the server, and optionally to display the blood glucose level.

Embodiment 36. A glucose monitoring system comprising: a wearable device capable of measuring blood glucose levels in a user, the device comprising: a housing body, an optical sensor comprising a circuit including a near infrared light emitting diode and a receiver chip and configured to produce a voltage signal, and a processing unit configured to convert the analog voltage signal to a digital voltage signal; and computer software comprising algorithms producing trained neural network models capable of predicting the user's blood glucose level in real time based on the voltage signals received from the processing unit, wherein the system is configured to non-invasively measure the user's blood glucose levels in real time, wherein the trained neural network models comprises a trained non-liner model and a linear model to execute the following steps: producing a class prediction probability value and a numerical value, by subjecting the voltage signals to the trained non-linear model and the linear model, respectively; classifying the class prediction probability value and the numerical value as being low, normal or high; comparing the classification results to determine if the values are consistent; and if the values are consistent, determining the output blood glucose state and the blood glucose value.

Embodiment 37. The glucose monitoring system of any one of the preceding embodiments, wherein the optical sensor is connected to the processing unit by the circuit.

Embodiment 38. The glucose monitoring system of any one of the preceding embodiments, wherein the trained neural network models are part of the processing unit.

Embodiment 39. The glucose monitoring system of any one of the preceding embodiments, wherein the trained neural network models are stored in a device or network separate from the device.

Embodiment 40. A method of monitoring blood glucose level, comprising the step of: (i) obtaining the first reflected light, the second reflected light and the third reflected light from the device as described in any one of the preceding embodiments, or the system as described in any one of the preceding embodiments; and (ii) calculating a blood glucose level based on the first reflected light, the second reflected light and the third reflected light.

Embodiment 41. The method of any one of the preceding embodiments, prior to step (ii), further comprising the step of: processing the first reflected light, the second reflected light and the third reflected light.

Embodiment 42. A method for processing data from a device or a system for blood glucose monitoring, comprising the steps of: a) obtaining the data vector from the device as described in any one of the preceding embodiments, or the system as described in any one of the preceding embodiments; b) pre-processing the data vector; and c) analyzing the data vector by trained machine learning module to produce an output data, such that a blood glucose level of the user is obtained.

1. A device for blood glucose monitoring of a user, comprising:
a) a light emitter, configured to emit a light signal directed to a target surface of the user, so as to generate a reflected light signal that is reflected from the target surface;
b) a light receiver, configured to receive the reflected light signals;
c) a controller, configured to operatively connect with the light emitter and the light receiver; and
d) an enclosure, configured to receive the light emitter, the light receiver and the controller,
wherein the light signal comprises a first light signal having a first wavelength of about 940 nm, a second light signal having a second wavelength of about 1350 nm, and/or a third light signal having a third wavelength of about 1500 nm,
wherein the controller comprises an operating module that controls the operation of the device, and converts the reflected light signal into a digital data, and
wherein the controller further comprises or operatively connects with a data processing system that processes the digital data, wherein the data processing system comprises a machine learning module that analyzes the data signal to generate an output data that is a blood glucose level of the user.

2. The device of claim 1, wherein the light emitter comprises three near infrared LEDs having the first wavelength, the second wavelength and the third wavelength, respectively.

3. The device of claim 1, wherein the device further comprises a NTC thermometer to obtain ambient temperature and/or the user's body temperature.

4. The device of claim 1, wherein the controller is further configured to control the light emitter to switch on and off to emit the first light signal, the second light signal and/or the third light signal back-to-back sequentially in a plurality of cycles, such that a plurality of light signal groups, each comprising the first reflected light signal, the second reflected light signal and/or the third reflected light signal obtained in each cycle are formed at a defined time interval.

5. The device of claim 4, wherein the time interval is about 60 times per minute.

6. The device of claim 4, wherein the controller comprises a processor unit coupled with a memory that stores an executable, software program, the software program comprises an operating module that controls the operation of the device, wherein the operation system executes the following steps:
a) controlling timing of the light emitter to switch on and off to emit the light signal;
b) controlling timing of light receiver to receive the reflected light signal to obtain a plurality of reflected light groups;
c) processing individual reflected light signal group into a digital, data vector; and
d) transmitting the data vector to the data processing system to analyze the data vector.

7. The device of claim 1, wherein the machine learning module comprises a deep meta learning framework (DMLF) module that processes the data vector obtained from the device to generate an output data.

8. The device of claim 1, wherein the data processing system executes the following steps:
a) obtaining the data vector from the device;
b) pre-processing the data vectors to produce processed data vector; and
c) analyzing the processed data vector by a trained DMLF module to produce an output data as a blood glucose level of the user.

9. The device of claim 8, wherein the step b) comprises the step of:
b1) cleaning the data vectors to remove any outlier;
optionally wherein the step b1) is performed by using isolation forest (iForest), one class SVM and LOF, a combination of isolation forest and one class SVM, or other combinations thereof.

10-11. (canceled)

12. The device of claim 8, wherein the step b) comprises the step of:
b2) consolidating a data vector into a representing data by the following equation:
representing data=(y+z)/2,
wherein y is the mean value of the data vector and z is the median value of the data vector.

13. The device of claim 7, wherein the DMLF module employs a deep meta-learning framework to analyze the data vector, comprising a first hierarchical layer and a second hierarchical layer, the first hierarchical layer comprising a plurality of basis models, and the second hierarchical layer comprising a meta model, wherein each basis models is configured to receive a processed data vector and produces an intermediate data point, and the meta model is configured to receive a weighted value of the intermediate data point to produce the output data;
optionally wherein the basis model is selected from the group consisting of RNN, LSTM, CNN, Support Vector Regression (SVR), MLP, KNN, ElasticNetCV, Catboost, XGBoost, Gradient Boosting Regressor, LGBM regressor, Bagging regressor, decision tree, XGBoost, and combinations thereof;
optionally wherein the meta model is selected from MLP, RNN, CNN, and any combination thereof;
optionally wherein the weighted value of the intermediate data point is computed by multiplying the intermediate data point by a model weight wherein each model weight is configured to be the same value, a random value or an optimized value obtained by a learning algorithm;
optionally wherein the first hierarchical layer comprises basis model of SVR, MLP and Catboost, and a meta model configured as a single layer MLP using ReLU as its activation function; and
optionally wherein the DMLF module is pre-trained by a set of training data using bootstrap sampling with a sampling with replacement methodology.

14-18. (canceled)

19. A device for blood glucose monitoring of a user, comprising:
a) a light emitter, having a plurality of near infrared LEDs, configured to emit a light signal directed to a target surface of the user, so as to generate a reflected light signal that is reflected from the target surface, respectively, wherein the light signal comprises a first light signal having a wavelength of about 940 nm, a second light signal having a wavelength of about 1350 nm and a third light signal having a wavelength of about 1500 nm;
b) a light receiver, having a photo-detector, configured to receive the reflected light signals; and
c) a controller, configured to operatively connect with the light emitter and the light receiver; and
d) an enclosure, configured to receive the light emitter, the light receiver and the controller, wherein the controller is configured to control the light emitter to switch on and off to emit the first light signal, the second light signal and the third light signal back-to-back sequentially, such that a plurality of reflected light groups, each comprising the first reflected light, the second reflected light and the third reflected light obtained in each cycle are formed at a defined time interval,
wherein the controller comprises a processor unit coupled with a memory that stores an executable, software program, the software program comprises an operating module that controls the operation of the device, and converts individual reflected light groups into a digital data vector,
wherein the controller further comprises or operatively connects with a data processing system that processes the data vector, wherein the data processing system comprises a machine learning module that analyzes the data vector to generate an output data that is a blood glucose level of the user;
wherein the software program optionally comprises an operating module that controls the operation of the device, wherein the operating module executes the following steps:
a) controlling timing of the light emitter to switch on and off to emit the first light, the second light and the third light;
b) controlling timing of light receiver to receive the first reflected light, the second reflected light and the third reflected light that are reflected from the target surface to generate a plurality of reflected light groups;
c) processing individual reflected light group into a digital, data vector; and
d) transmitting the data vector to a data processing system comprising a neural network to process the data vectors; and
optionally wherein the machine learning module comprises a deep meta learning framework module, comprising a first hierarchical layer and a second hierarchical layer, the first hierarchical layer comprises a plurality of basis modules, the second hierarchical layer comprises a meta learning module, wherein each basis module is configured to receive a processed data vector and produces an intermediate data point, the meta learning module is configured to receive a weighted value of the intermediate data point to produce the output data, wherein first hierarchical layer comprises basis modules of SVR, MLP and Catboost, and second hierarchical layer comprises a meta learning module of ReLU.

20-21. (canceled)

22. A system for blood glucose monitoring of a user, comprising:
a) a device as claimed in claim 1; and
b) a server in electrical communication with the device.

23. The system of claim 22, wherein the server comprises a server processor unit coupled with a server memory that stores an executable server software program, the server software program comprises a data processing system that processes a data vector obtained from the device to calculate the blood glucose level of the user, wherein the data processing system comprises a neural network.

24-34. (canceled)

35. The system of claim 22, further comprising a mobile apparatus that is in electrical communication between the device and the server, configured to receive a data vector obtained from the device, to transmit the data vector to the server, and optionally to display the blood glucose level.

36. The system of claim 22, further comprising:
a housing body, an optical sensor comprising a circuit including a near infrared light emitting diode and a receiver chip and configured to produce a voltage signal, and a processing unit configured to convert the analog voltage signal to a digital voltage signal; and computer software comprising algorithms producing trained neural network models capable of predicting the user's blood glucose level in real time based on the voltage signals received from the processing unit, wherein the system is configured to non-invasively measure the user's blood glucose levels in real time, wherein the trained neural network models comprises a trained non-liner model and a linear model to execute the following steps:
producing a class prediction probability value and a numerical value, by subjecting the voltage signals to the trained non-linear model and the linear model, respectively;
classifying the class prediction probability value and the numerical value as being low, normal or high;
comparing the classification results to determine if the values are consistent; and
if the values are consistent, determining the output blood glucose state and the blood glucose value.

37. The glucose monitoring system of claim 36, wherein the optical sensor is connected to the processing unit by the circuit.

38. The glucose monitoring system of claim 36, wherein the trained neural network models are part of the processing unit.

39. The glucose monitoring system of claim 36, wherein the trained neural network models are stored in a device or network separate from the device.

40. A method of monitoring blood glucose level, comprising the steps of:
(i) obtaining the first reflected light, the second reflected light and the third reflected light from the device as claimed in claim 1; and
(ii) calculating a blood glucose level based on the first reflected light, the second reflected light and the third reflected light; wherein
optionally prior to step (ii), further comprising the step of:
processing the first reflected light, the second reflected light and the third reflected light.

41-42. (canceled)
